(19) United States
(12) Patent Application Publication
(10) Pub. No.: US 2013/0115640 A1
(43) Pub. Date: May 9, 2013

(54) ACTH for Treatment of Kidney Disease

(71) Applicants: James A. Tumlin, Chattanooga, TN (US); Claude Galphin, Chattanooga, TN (US); Brad Rovin, Columbus, OH (US)

(72) Inventors: James A. Tumlin, Chattanooga, TN (US); Claude Galphin, Chattanooga, TN (US); Brad Rovin, Columbus, OH (US)

(21) Appl. No.: 13/667,945

(22) Filed: Nov. 2, 2012

Related U.S. Application Data
(60) Provisional application No. 61/555,432, filed on Nov. 3, 2011.

(51) Int. Cl. G01N 33/68; G06F 19/00

(52) U.S. Cl. CPC G01N 33/6893; G06F 19/34
USPC 435/7.94; 435/7.92; 435/287.2; 702/19

(57) ABSTRACT

Provided herein are methods for prophylactic treatment of Diabetes Mellitus comprising administration of adrenocorticotropic hormone (ACTH), or fragment, analog, complex or aggregate thereof, or any combination thereof, to an individual suspected of having, predisposed to, or at risk of developing Diabetes Mellitus. Also provided herein are methods for monitoring treatment of Diabetes Mellitus by measuring and monitoring levels of MCP-1, TGF-β, VEGF A and VEGF B.

### CROSS REFERENCE

This application claims the benefit of U.S. Provisional Application No. 61/555,432, filed Nov. 3, 2011, which application is incorporated herein by reference in its entirety.

### BACKGROUND OF THE INVENTION

Diabetic nephropathy is a frequent and serious complication of long-term diabetes and is now the leading cause of end-stage renal disease (ESRD) in the United States and other developed countries [1]. In the United States there is a growing incidence of diabetes with approximately 17 million people, or 6.2% of the population now estimated to have the disease. Roughly one third of this population is estimated to be undiagnosed with type II diabetes. The prevalence of diabetes is higher in certain racial and ethnic groups, affecting approximately 13% of African Americans, 10.2% of Hispanics, and 15.1% of Native Americans, primarily with type II diabetes. Approximately 20% to 30% of all diabetics will develop evidence of nephropathy, although a higher percentage of type I patients progresses to ESRD. The incidence of diabetic nephropathy varies between demographic groups, but approximately 40% of patients with type I diabetes will develop progressive disease with the majority of the patient reaching ESRD within 10 years. In the United Kingdom, the Prospective Diabetes Study Group attempted to determine the incidence of progressive diabetic nephropathy by prospectively following 2,856 patients with newly diagnosed diabetes. At study entry, 18% of patients had microalbuminuria while 3% had microalbuminuria.

### SUMMARY OF THE INVENTION

Monitoring treatment of diabetes is crucial to long-term health of the patient. Without careful disease management, a patient's symptoms can quickly deteriorate leading to end stage renal disease and, ultimately, death of the patient due to organ failure. Reliable and fast methods of assessing a patient for initial treatment, for stopping treatment, or for managing the dose and/or duration of treatments of patients undergoing ACTH therapy are needed. As used herein, the term “adrenocorticotropic hormone (ACTH)” refers to ACTH(1-39), fragments thereof, and analogs thereof as described in more detail below. The present inventors have identified for the first time that the combination of urinary VEGF 121 (VEGF A), VEGF 165 (VEGF B) and MCP-1 represent a reliable series of markers for managing patient care.

Provided herein is a method of identifying a patient for treatment with ACTH(1-39), a fragment thereof, or an analog thereof, and/or managing the dose and/or duration of ACTH treatment in the long-term management of diabetic nephropathy or nephrotic range proteinuria in a patient comprising: measuring levels of urinary VEGF 121, VEGF 165 and MCP-1 in one or more urine samples obtained from said patient; and adjusting the dose and/or duration of ACTH(1-39), a fragment thereof, or an analog thereof, administered to said patient based on the levels of said VEGF 121, VEGF 165 and MCP-1. ACTH may be administered, for example, as Acthar® gel.

When the ratio of VEGF 121 to VEGF 165 is below about 0.80, a patient begins treatment, or the dose is increased or the duration of treatment is increased. In one embodiment, the ratio is below about 0.75, below about 0.70, below about 0.65, below about 0.60, below about 0.55, or below about 0.50. In another embodiment, the ratio is below 0.80, below 0.75, below 0.70, below 0.65, below 0.60, below 0.55, or below 0.50.

A patient begins treatment, or alternatively, the dose and/or duration of treatment of a patient may be increased if the concentration of urinary VEGF 121 is greater than about 200 pg/mg Cr, about 225 pg/mg Cr, about 250 pg/mg Cr, about 500 pg/mg Cr, about 750 pg/mg Cr, about 1000 pg/mg Cr, about 1250 pg/mg Cr, about 1500 pg/mg Cr, about 1750 pg/mg Cr, about 2000 pg/mg Cr, or more. In one embodiment, the concentration of urinary VEGF 121 is greater than 200 pg/mg Cr, about 225 pg/mg Cr, 250 pg/mg Cr, 500 pg/mg Cr, 750 pg/mg Cr, 1000 pg/mg Cr, 1250 pg/mg Cr, 1500 pg/mg Cr, 1750 pg/mg Cr, 2000 pg/mg Cr, or more. In another embodiment, the concentration of urinary VEGF 121 is greater than about 200 pg/mg Cr, or is greater than 200 pg/ml Cr.

A patient begins treatment, or alternatively, the dose and/or duration of treatment of a patient may be increased if the concentration of urinary VEGF 165 is greater than about 160 pg/ml, about 180 pg/mg Cr, about 200 pg/mg Cr, about 225 pg/mg Cr, about 250 pg/mg Cr, about 500 pg/mg Cr, about 750 pg/mg Cr, about 1000 pg/mg Cr, about 1250 pg/mg Cr, about 1500 pg/mg Cr, about 1750 pg/mg Cr, about 2000 pg/mg Cr, or more. In one embodiment, the concentration of urinary VEGF 165 is greater than 160 pg/ml, 180 pg/mg Cr, 200 pg/mg Cr, 225 pg/mg Cr, 250 pg/mg Cr, 500 pg/mg Cr, 750 pg/mg Cr, 1000 pg/mg Cr, 1250 pg/mg Cr, 1500 pg/mg Cr, 1750 pg/mg Cr, 2000 pg/mg Cr, or more. In another embodiment, the concentration of urinary VEGF 165 is greater than about 160 pg/mg Cr or greater than 160 pg/ml Cr.

A patient begins treatment, or alternatively, the dose and/or duration of treatment of a patient may be increased if the concentration of urinary MCP-1 is greater than about 0.25 ng/mg, about 0.50 ng/ml, or about 0.75 ng/ml.

The method of claim 1, wherein if the concentration of urinary MCP-1 is less than about 0.25 ng/mg, about 0.20 ng/ml, about 0.175 ng/ml, about 0.15 ng/ml, about 0.125 ng/ml or less, treatment of the patient is stopped, or the dose and/or duration of treatment is decreased.

Alternatively, a patient begins treatment, or alternatively, the dose and/or duration of treatment of a patient may be increased if the concentration of urinary MCP-1 is less than about 1000 mg/24 hours. In one embodiment, the concentration of urinary MCP-1 is less than about 900 mg/24 hours, about 800 mg/24 hours, about 750 mg/24 hours, about 700 mg/24 hours, about 600 mg/24 hours, about 500 mg/24 hours, or less. In another embodiment, the concentration of urinary MCP-1 is less than 900 mg/24 hours, 800 mg/24 hours, 750 mg/24 hours, 700 mg/24 hours, 600 mg/24 hours, 500 mg/24 hours, or less.

Treatment of a patient may cease, or alternatively, the dose and duration of treatment of a patient may be decreased if the concentration of urinary MCP-1 is greater than about 1000 mg/24 hours. In one embodiment, the concentration of urinary MCP-1 is greater than about 1250 mg/24 hours, about 1500 mg/24 hours, about 1750 mg/24 hours, about 2000 mg/24 hours, or more. In another embodiment, the concentration of urinary MCP-1 is greater than 1250 mg/24 hours, 1500 mg/24 hours, 1750 mg/24 hours, 2000 mg/24 hours, or more.

In one aspect of such methods, the concentration of urinary VEGF 121 may be greater than about 200 pg/mg Cr, the concentration of urinary VEGF 165 may be greater than about 160 pg/mg Cr, and the concentration of urinary MCP-1 may be greater than about 0.25 ng/mg, and if the ratio of VEGF 121 to VEGF 165 is below about 0.80, a patient begins treatment, the dose is increased or the duration of treatment is increased.

In another aspect of such methods, the concentration of urinary VEGF 121 may be less than about 200 pg/mg Cr, the concentration of urinary VEGF 165 may be less than about 160 pg/mg Cr, the concentration of urinary MCP-1 may be less than about 0.25 ng/mg, and if the ratio of VEGF 121 to VEGF 165 is above about 0.80, a patient ceases treatment, the dose is decreased or the duration of treatment is decreased.

In another aspect, each dose of ACTH(1-39), a fragment thereof, or an analog thereof, may be increased if a patient is identified as needing further treatment. In one embodiment, a dose concentration of ACTH(1-39), a fragment thereof, or an analog thereof, is increased by about 1 unit, about 2 units, about 3 units, about 4 units, about 5 units, about 6 units, about 7 units, about 8 units, about 9 units, about 10 units, about 12 units, about 14 units or 16 units compared to existing treatment doses of the patient. In one non-limiting example, a dose concentration of 16 units of ACTH(1-39), a fragment thereof, or an analog thereof, may be increased to about 18 units, about 20 units, about 22 units, about 24 units, about 26 units, or more, one or more doses are administered to a patient undergoing treatment. In another embodiment, a dose concentration of 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg may be increased by about 10 mg/kg, where one or more doses are administered to a patient undergoing treatment. In yet another embodiment, each dose of ACTH(1-39), a fragment thereof, or an analog thereof, administered to said patient may be increased by about 5%, by about 10%, by about 15%, by about 20%, by about 25%, by about 30%, by about 40%, by about 50% or more. Additional doses and administrations that may be utilized are described herein in more detail below. Compositions for administration are also described herein in more detail below.

Where a patient is identified for increased treatment, the frequency of ACTH(1-39), a fragment thereof, or an analog thereof, administration may be increased until the patient responds. For example, a treatment regimen of daily administration of ACTH(1-39), a fragment thereof, or an analog thereof, for 6 months may extended to about 7 months, about 8 months, about 9 months, about 10 months, about 11 months, about 12 months, about 13 months, about 14 months, about 15 months, about 16 months, about 17 months, about 18 months, about 2 years, about 3 years, about 4 years, about 5 years or more.

Where a patient is receiving a once-daily dose of ACTH(1-39), a fragment thereof, or an analog thereof, an increase may include a twice-daily administration of ACTH(1-39), a fragment thereof, or an analog thereof.

One would understand that intervals of treatment for patients in remission may be altered as needed if the patient is identified as needing further treatment using a method described herein. For example, in one instance in which a patient in remission was receiving a dose once per month would start receiving a dose every one or two weeks, or depending upon the levels of biomarkers, return to a once daily treatment.

The dosage of ACTH(1-39), a fragment thereof, or an analog thereof, treatment may be decreased if a patient is identified as successfully responding to treatment. In one embodiment, a dose concentration of ACTH(1-39), a fragment thereof, or an analog thereof, may be decreased by about 1 unit, about 2 units, about 3 units, about 4 units, about 5 units, about 6 units, about 7 units, about 8 units, about 9 units, about 10 units, about 12 units, about 14 units or 16 units compared to existing treatment doses of the patient. For example, a dose concentration of 16 units of ACTH(1-39), a fragment thereof, or an analog thereof, may be decreased to about 14 units, about 12 units, about 10 units, about 8 units, or less.

In another embodiment, a dose concentration of 10 mg/kg, 20 mg/kg, 30 mg/kg, 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg or about 200 mg/kg may be decreased by about 5-10 mg/kg. For example, a dose concentration of 150 mg/kg may be decreased to about 145 mg/kg or 140 mg/kg. Alternatively, the dose of ACTH(1-39), a fragment thereof, or an analog thereof, administered to said patient may be reduced by about 5%, about 10% by about 15%, by about 20%, by about 25%, by about 30%, by about 40%, by about 50% or more.

ACTH(1-39), a fragment thereof, or an analog thereof, treatment may be decreased if a patient is identified as successfully responding to treatment. In one embodiment, the duration of ACTH(1-39), a fragment thereof, or an analog thereof, administration is reduced from once every day to once every two days, every three days, every 4 days, every 5 days, every 6 days, every 7 days, every 1.5 weeks, every 2 weeks, every 3 weeks, every 4 weeks, once every 2 months, once every 4 months, once every 6 months or once per year.

One would understand that treatment may be modified in any number of ways: by dose, frequency, intervals of treatment, or a combination thereof. For example, in one instance, a patient having received treatment with ACTH(1-39), a fragment thereof, or an analog thereof, may receive daily treatment for 6-12 months and, thereafter, receive a once-weekly or a once-monthly dose for one, two, three, four, five or more years. In another example, a once daily or twice daily dose regimen is increased to once per week, once every two weeks, once per month, once every two months, once every three months, once every 6 months or more. Treatment can be modified based upon the methods of monitoring described herein.

Urine samples may be obtained to determine a baseline level of proteins, prior to treatment, at one or more times during treatment, and at one or more times post-treatment. Protein levels may be compared to a standard concentration, to levels of proteins from healthy patients and/or to levels of proteins from sick patients.

One or more additional proteins may be measuring and the results thereof utilized to determine whether or not a patient needs to start treatment, increase the dose and/or duration of treatment, stop treatment, or decrease the dose and/or duration of treatment. Non-limiting examples of proteins to be tested include, but are not limited to, urinary TGF-β, urinary creatine, or both. Proteinuria may also be measured and used in the assessments.

Provided herein is a method of decreasing glomerular permeability in a patient, comprising measuring levels of urinary VEGF 121, VEGF 165 and MCP-1 in one or more urine samples obtained from said patient, wherein if the concentration of urinary VEGF 121 is greater than about 200 pg/mg Cr, the concentration of urinary VEGF 165 is greater than about 160 pg/mg Cr; and the concentration of urinary MCP-1 is 250 pg/mg or less than about 1000 mg/24 hours, the dose and/or duration of ACTH(1-39), a fragment thereof, or an analog thereof, administered to said patient is increased.

In one embodiment, rising levels (concentrations) of VEGF 121 and MCP-1 and decreasing levels of VEGF 165 as biomarkers may indicate increasing glomerular permeability, and the dose and/or duration of ACTH(1-39), a fragment thereof, or an analog thereof, treatment is increased.

In another embodiment, decreasing levels of VEGF 121 and MCP-1 and increasing levels of VEGF 165 as biomarkers may indicate decreasing glomerular permeability, and the dose and/or duration of ACTH(1-39), a fragment thereof, or an analog thereof, treatment is decreased.

In another embodiment, decreasing levels of VEGF 121 and increasing levels of VEGF 165 as biomarkers may indicate decreasing glomerular permeability, and the dose and/or duration of ACTH(1-39), a fragment thereof, or an analog thereof, treatment is decreased.

Provided herein is a method of decreasing proteinuria in a patient, comprising measuring levels of urinary VEGF 121, VEGF 165 and MCP-1 in one or more urine samples obtained from said patient, wherein if the ratio of VEGF 121 to VEGF 165 falls below about 0.80, proteinuria is diagnosed as increasing, and the dose and/or duration of ACTH(1-39), a fragment thereof, or an analog thereof, administered to said patient is increased.

Provided herein is a method of identifying a patient for treatment with ACTH(1-39), a fragment thereof, or an analog thereof, and/or increasing the dose and/or duration of ACTH(1-39), a fragment thereof, or an analog thereof, treatment in the long-term management of diabetic nephropathy or nephrotic range proteinuria in a patient comprising measuring levels of urinary VEGF 121 of between about 200 pg/ml and 800 pg/ml, urinary levels of VEGF 165 of between about 180 pg/ml and 2000 pg/ml, and urinary levels of MCP-1 of between about 0.25 ng/mg or 400 mg/24 hours to about 0.4 ng/mg or 1000 mg/24 hours in one or more samples obtained from said patient, identifying a ratio of VEGF 121 to VEGF 165 of below about 0.80, and beginning treatment of said patient or increasing the dose and/or duration of ACTH administered to said patient based on the levels of said VEGF 121, VEGF 165 and MCP-1.

Provided herein is a method of identifying a patient for treatment with ACTH(1-39), a fragment thereof, or an analog thereof, and/or increasing the dose and/or duration of ACTH(1-39), a fragment thereof, or an analog thereof, treatment in the long-term management of diabetic nephropathy or nephrotic range proteinuria in a patient comprising identifying levels of urinary VEGF 121 of least 200 pg/ml, urinary levels of VEGF 165 of at least 180 pg/ml and urinary levels of MCP-1 of less than about 1000 mg/24 hours or 0.25 ng/mg in one or more samples obtained from said patient and beginning treatment of said patient or increasing the dose and/or duration of ACTH(1-39), a fragment thereof, or an analog thereof, administered to said patient based on the levels of said VEGF 121, VEGF 165 and MCP-1.

Provided herein is a method of identifying a patient for cessation of treatment with ACTH(1-39), a fragment thereof, or an analog thereof, and/or decreasing the dose and/or duration of ACTH(1-39), a fragment thereof, or an analog thereof, treatment in the long-term management of diabetic nephropathy or nephrotic range proteinuria in a patient comprising measuring levels of urinary VEGF 121 of below about 200 pg/ml, urinary levels of VEGF 165 of below about 180 pg/ml, and urinary levels of MCP-1 of greater than about 0.25 ng/mg or less than about 1000 mg/24 hours in one or more samples obtained from said patient; measuring a ratio of VEGF 121 to VEGF 165 of above about 0.80; and ceasing treatment of said patient or decreasing the dose and/or duration of ACTH(1-39), a fragment thereof, or an analog thereof, administered to said patient based on the levels of said VEGF 121, VEGF 165 and MCP-1.

Provided herein is a method of identifying a patient for treatment with ACTH(1-39), a fragment thereof, or an analog thereof, and/or increasing the dose and/or duration of ACTH(1-39), a fragment thereof, or an analog thereof, treatment in the long-term management of diabetic nephropathy or nephrotic range proteinuria in a subject comprising: measuring levels of urinary VEGF 121, VEGF 165 and MCP-1 in urine samples obtained from said patient, wherein if the ratio of VEGF 121 to VEGF 165 is below about 0.80 and if the level of urinary MCP-1 is less than about 1000 mg/24 hours or above about 0.25 ng/mg, the patient is identified for further treatment, and beginning treatment of said patient or increasing the dose and/or duration of ACTH(1-39), a fragment thereof, or an analog thereof, treatment.

Provided herein is a method of identifying a patient for cessation of treatment with ACTH(1-39), a fragment thereof, or an analog thereof, and/or decreasing the dose and/or duration of ACTH(1-39), a fragment thereof, or an analog thereof, treatment in the long-term management of diabetic nephropathy or nephrotic range proteinuria in a subject comprising measuring levels of urinary VEGF 121, VEGF 165 and MCP-1 in urine samples obtained from said patient, wherein if the ratio of VEGF 121 to VEGF 165 is above about 0.80 and if the level of urinary MCP-1 is greater than about 1000 mg/24 hours or below about 0.25 ng/mg, the patient is identified as needing reduced treatment; and ceasing treatment of said patient or decreasing the dose and/or duration of ACTH(1-39), a fragment thereof, or an analog thereof, treatment. In one embodiment, the patient is identified as being in remission.

Provided herein is a method of reducing end-organ expression of melanocortin receptors and downstream loss of VEGF B production in a patient in need thereof, comprising identifying a patient in need of treatment according to the methods described above; and administering ACTH(1-39), a fragment thereof, or an analog thereof, to said patient, wherein downstream loss of VEGF B production is inhibited.

Provided herein is a method of treating patient subject suffering from nephrotic range proteinuria, comprising identifying a patient in need of treatment according to the methods described above; and administering to said patient one or more doses of an effective amount of ACTH(1-39), a fragment thereof, or an analog thereof, whereby said patient is partially or completely treated.

Provided herein is a method of inhibiting podocyte failure/degradation in a patient diagnosed with nephrotic range proteinuria comprising identifying a patient in need of treatment according to the methods described above, and administering an effective treatment regimen of ACTH(1-39), a fragment thereof, or an analog thereof, whereby podocyte failure/degradation in said patient is inhibited.

Provided herein is a method of reducing nephritic range proteinuria in a patient having CKD stage II/III diabetic nephropathy comprising identifying a patient in need of treatment according to the methods described above; and administering one or more doses of an effective amount of ACTH(1-39), a fragment thereof, or an analog thereof, whereby the concentration of urinary VEGF 165 is increased to at least about 180 pg/ml, and whereby nephritic range proteinuria in a patient is reduced.

Provided herein is a system for identifying a patient for treatment with ACTH(1-39), a fragment thereof, or an analog thereof, and/or managing the dose, interval and/or duration of ACTH treatment in the long-term management of diabetic nephropathy or nephrotic range proteinuria in a patient comprising antibodies that specifically bind to urinary VEGF 121, VEGF 165 and MCP-1; an optical density microplate reader; and a composition comprising ACTH(1-39), a fragment thereof, or an analog thereof. In one embodiment, the system further comprises an optionally networked computer processing device configured to perform executable instructions; and a computer program, the computer program comprising a software module executed by the computer processing device to apply a model or algorithm for analyzing the urinary levels of VEGF 121, VEGF 165 and MCP-1 in the sample.

Provided herein is a computer-implemented system comprising: (a) a computer comprising: a processor, an operating system configured to perform executable instructions, and a memory device; and (b) a computer program including instructions executable by the computer, the program comprising (i) a software module configured to receive data indicating levels of urinary VEGF 121, VEGF 165 and MCP-1 in one or more urine samples obtained from a patient, the patient in need of long-term management of diabetic nephropathy or nephrotic range proteinuria; (ii) a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and (iii) a software module configured to generate a report comprising a recommendation for an adjustment to the dose, interval and/or duration of ACTH(1-39), a fragment thereof, or an analog thereof administered to the patient.

In one embodiment, the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

In another embodiment, the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.

Also provided herein is a non-transitory computer-readable storage media encoded with a computer program including instructions executable by a processor, the program comprising (a) a software module configured to receive data indicating levels of urinary VEGF 121, VEGF 165 and MCP-1 in one or more urine samples obtained from a patient, the patient in need of long-term management of diabetic nephropathy or nephrotic range proteinuria; (b) a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and (c) a software module configured to generate a report comprising a recommendation for an adjustment to the dose, interval and/or duration of ACTH(1-39), a fragment thereof, or an analog thereof administered to the patient.

In one embodiment, the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

In another embodiment, the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the embodiments are set forth with particularity in the appended claims. A better understanding of the features and advantages of the present embodiments will be obtained by reference to the following detailed description that sets forth illustrative embodiments, in which the principles of the embodiments are utilized, and the accompanying drawings of which:

FIG. 1 provides an exemplary screening protocol for the study described in the Examples.

FIG. 2 provides the screening strategy and inclusion exclusion criteria for Acthar® Gel Treatment of Nephrotic Range Diabetic Nephropathy.

FIG. 3A provides the patient demographics for the ACTH-Diabetic Nephropathy Trial. FIG. 3B shows the numbers of patients screened and tested throughout the study of Example 1.

FIG. 4 illustrates urinary protein levels (mg/24 hours) data for patients receiving therapy of 16 units of Acthar® Gel at times of pre-treatment (baseline), after 6 months of taking Acthar® Gel, after 6 months post-therapy, and at 12 months post-therapy. Five (5) of 7 patients exhibited a complete-partial response and 3 of 7 patients were still in remission 12-months post-therapy.

FIG. 5 illustrates urinary protein levels (mg/24 hours) data for patients receiving therapy of 32 units of Acthar® Gel at times of pre-treatment (baseline), after 6 months of taking Acthar® Gel, after 6 months post-therapy, and at 12 months post-therapy. Three (3) of 7 patients exhibited a complete-partial response and 1 of 7 patients was still in remission 12-months post-therapy.

FIG. 6 illustrates pre-post urine levels of VEGF using an ELISA assay after treatment with 16 units of Acthar® Gel.

### DETAILED DESCRIPTION OF THE INVENTION

### Diabetes Mellitus

Diabetes Mellitus is a significant and growing health problem in the United States and other developed countries. Despite improving public awareness, end-organ complications including diabetic nephropathy and coronary atherosclerotic heart disease continues to grow by 5-10% per year. While improvements in the control of blood pressure and the wide-spread use of antagonists of the renin-angiotensin-aldosterone system have significantly improved renal outcomes, therapies designed to disrupt the more central pathogenic mechanisms of diabetic nephropathy are still needed. Recent observations have shown that effacement of podocyte foot-plate processes and accelerated apoptosis are involved in the pathogenesis of diabetic nephropathy. The resulting increase in glomerular permeability leads to nephrotic range proteinuria and interstitial fibrosis from local synthesis of transforming growth factor β (TGF-β) and direct toxicity to the renal epithelium.

Recent studies have shown that synthetic forms of adrenocorticotropic hormone (ACTH) are able to achieve sustained reductions in proteinuria in diabetic nephropathy. The observation that ACTH can reduce proteinuria in diabetes as well as other forms of glomerulopathies led the present inventors to consider that the site of effect of ACTH may involve the podocyte. Type 1 Melanocortin receptors are expressed in the glomerular podocyte.

Current studies have not been able to delineate the mechanisms by which ACTH is able to improve glomerular function, but there may be a link between hyperglycemia, increased production of transforming growth factor beta (TGF-β) and glomerular expression of ACTH receptors. For example, studies in adrenal cortical cells suggest that TGF-β is able to down regulate the basal and autocrine induced expression of ACTH receptors. This unexpected link between TGF-β and ACTH signaling raises the question of whether impaired signaling of ACTH leads to increased glomerular permeability through podocyte apoptosis and detachment.

The present inventors examined whether the protein lowering effects of ACTH in patients with diabetic nephropathy may involve increased production of VEGF.

The data presented in this application demonstrates for the first time that ACTH can induce a complete or partial response in reducing proteinuria in 8 of 14 patients and that a rise in urinary VEGF occurred in all responsive patients.

Pathogenesis: Role of Podocyte Dysfunction and Propagation of Diabetic Nephropathy:

The pathologic hallmarks of diabetic nephropathy include expansion of mesangial matrix, thickening of the basement membrane and formation of glomerular sclerotic lesions. These hallmarks are now thought to be extensions of a central disorder within the visceral epithelial cells or podocytes. Specifically, mesangial matrix expansion correlates closely with both proteinuria and deterioration of renal function.

It has been proposed that accumulation of matrix in the mesangial area reduces the capillary surface area available for filtration, thereby contributing to the progressive loss of viable nephrons. However, expansion and thickening of the mesangial matrix does not explain the increased permeability to proteins that is a clinical hallmark of diabetic nephropathy. Indeed, a thickened, denser matrix would be more likely to explain a reduction in glomerular filtration rather than increase in permeability. These observations have directed the clinical research efforts away from the mesangium and basement membrane and onto the glomerular podocyte. For example, early work in diabetic rats demonstrated that foot processes of the podocyte were widened in animals with both diabetic and non-diabetic forms of proteinuria [15].

More recently, clinical observations have confirmed animal models. Berg et al. examined renal biopsies in patients with type I diabetic nephropathy and noted that the degree of foot plate effacement correlated with the urinary albumin excretion rates [5; 6]. In addition to the widening of foot processes, Steffes et al. noted that the actual number glomerular podocytes falls in patients with progressive diabetic nephropathy [16; 14; 15]. Indeed, the clinical histopathologic finding that is most predictive of patients with progressive diabetic nephropathy was that the change in glomerular podocyte density was the strongest predictor of progressive renal disease [13]. In addition to disorders with foot plate extensions, podocytes in patients with diabetic nephropathy also express reduced cell adherence to the basement membrane [13]. Because the loss of podocyte into the urinary space correlates with the degree of cellular injury, Nakamura et al. has examined whether podocyte markers can be used as mean for monitoring clinical disease.

The observation that ACTH can have profound effects on proteinuria in patients with advanced diabetes, suggests that ACTH may have unrecognized regulatory or trophic effects upon the glomerular podocyte [4].

The present application addresses the efficacy of Acthar® Gel to reduce nephrotic range proteinuria in patients with CKD stage II/III diabetic nephropathy and a new combination of markers for better managing treatment of patients.

Pathogenic Role of TGF-β1 in Diabetic Nephropathy:

The extracellular environment is composed of a complex matrix of carbohydrates, glycoproteins and structural proteins including integrins, fibronectin, laminin and up to four different forms of collagen. While these glycoproteins are a normal part of renal physiology, repeated injury (whether through hypertension, diabetes or chronic inflammation) leads to expansion of the extracellular matrix environment. Interestingly, tubular atrophy and interstitial fibrosis appears to be a common byproduct of renal injury and independent of the original insult.

Thus, pharmacologic maneuvers designed to block fibrotic pathways could reduce the loss of nephron mass over time and, ultimately, slow the progression toward ESRD.

Indeed, previous clinicopathologic studies in diabetic nephropathy, hypertensive nephrosclerosis and other forms of glomerular injury find that the degree of interstitial fibrosis is the most predictive variable for identifying patients at risk for progressive renal disease. These observations have led investigators to determine the role of transforming growth factor beta (TGF-β) and other cytokines involved in chronic wound healing in the pathogenesis of diabetic nephropathy.

The superfamily of transforming growth factors are expressed in virtually all cell types and mediate both pro-inflammatory responses and the deposition of extra cellular matrix proteins in chronic wound healing. Three isoforms of TGF-β are known to exist which bind to classic heterotrimeric receptor kinases and initiate a complex signaling pathway leading to the phosphorylation and activation of the SMAD transcription factors. Following migration into the nucleus, activated SMAD proteins stimulates TGF-specific genes involved in growth & differentiation, apoptosis, regulation of immune processes. Moreover, TGF-β central to the transcription of proteins involved in chronic wound healing including type III/IV collagen, Laminin, fibronectin and other extra-cellular matrix proteins [17]. TGF-β is stimulated by conditions of hyperglycemia [7]. For examples, micropuncture studies in animal models of streptozosin-induced diabetes demonstrate that TGF-β is expressed within the glomerulus and then is released into the proximal tubule. Moreover, neutralizing TGF-β with a pan-selective neutralizing antibody blocks the renal hypertrophy, expansion of the mesangial matrix and the development of renal insufficiency.

Over-expression of TGF-β1 in non-diabetic animals leads to podocyte foot-plate effacement and increased albumin permeability. The present inventors identified that the effects of TGF-β in the podocyte may not be unique to diabetes and that podocyte dysfunction may be central to a variety of different glomerular disorders.

### Biomarkers: Urinary VEGF A (VEGF 121), VEGF B (VEGF 165) and MCP-1; Clinical Utility in Management of Diabetic Nephropathy

The present inventors have identified a new combination of markers that allow earlier identification and treatment of diabetic nephropathy or nephrotic range proteinuria as well as prevent progression of diabetic nephropathy or nephrotic range proteinuria. In some embodiments of the methods described herein, selection of an individual suspected of having, predisposed to, or at risk of developing a diabetic nephropathy or nephrotic range proteinuria is through the use of biomarkers thereby initiating a prophylactic treatment regimen. In other embodiments of the methods described herein, the efficacy of treatment of a patient diagnosed with diabetic nephropathy or nephrotic range proteinuria may be monitored through the use of biomarkers.

Inflammatory cytokines have also been linked to progression in diabetic nephropathy. Monocyte chemoattractic factor-1 (MCP-1) is a 30 kD glycosylated protein that is secreted following tissue injury. When bound to its receptor protein (CCR2), MCP-1 stimulates the attraction of circulating monocytes and tissue macrophages to areas of injury. Many of the clinical consequences of diabetes including high glucose, advanced glycosylated products and reactive oxidant stress [23] can stimulate production of MCP-1 by tubulointerstitial cells within the kidney. The possible tubulointerstitial source of MCP-1 has been observed in other glomerulopathies in which high grade proteinuria itself has been suggested to initiate interstitial inflammation and lead to progressive fibrosis [23].

Levels of MCP-1 in the urine have been found to be elevated in patients with diabetic nephropathy and, thus, it may be used as a marker of disease progression. For example, urinary MCP-1 levels also fall with prolonged ACE inhibition and correlate with reduced progression of diabetic nephropathy. These observations suggest that a potential benefit of ACTH on progression of diabetic nephropathy may involve a reduction in local MCP-1 production initiated by the development of macroalbuminuria. ACTH has also been found able to block MCP-1 production through its inhibitory effects on NF-Kb.

As used herein the term “vascular endothelial growth factor (VEGF)”, also known as vascular permeability factor (VPF), is a potent mediator of both angiogenesis and vasculogenesis in the fetus and adult. It is a member of the PDGF family that is characterized by the presence of eight conserved cysteine residues in a cystine knot structure and the formation of anti-parallel disulfide-linked dimers. Humans express alternately spliced isoforms of 121, 145, 165, 183, 189, and 206 amino acids in length. VEGF 165 (VEGF B) appears to be the most abundant and potent isoform, followed by VEGF 121 and VEGF 189. Isoforms other than VEGF 121 contain basic heparin-binding regions and are not freely diffusible. Human VEGF 165 shares 88% amino acid sequence identity with corresponding regions of mouse and rat VEGF. VEGF is expressed in multiple cells and tissues including skeletal and cardiac muscle, hepatocytes, osteoblasts, neutrophils, macrophages, keratinocytes, brown adipose tissue, CD34+ stem cells, endothelial cells, fibroblasts, and vascular smooth muscle cells. VEGF plays a role in vasculogenesis. During embryogenesis, VEGF regulates the proliferation, migration, and survival of endothelial cells, thus regulating blood vessel density and size, but playing no role in determining vascular patterns. VEGF promotes bone formation through osteoblast and chondroblast recruitment and is also a monocyte chemoattractant. In postnatal life, VEGF maintains endothelial cell integrity and is a potent mitogen for micro- and macro-vascular endothelial cells. In adults, VEGF functions mainly in wound healing and the female reproductive cycle. In diseased tissues, VEGF promotes vascular permeability.

The present inventors have identified for the first time that the combination of urinary VEGF 165, MCP-1 and VEGF 121 may be used for clinical management of diabetic nephropathy or nephrotic range proteinuria. With the combination of biomarkers provided herein, early detection, predisposition and diagnosis can be enhanced for diabetic nephropathy or nephrotic range proteinuria as well as monitoring the time frame for administering prophylactic therapies to a subject.

TGFβ may also be utilized as an additional marker in the methods described herein. As used herein, the term “transforming growth factor (TGF),” refers to a protein that is a member of a superfamily that includes TGF-β1 through 5, bone morphogenic proteins, activins and inhibins. Human TGF-β1 is a 25 kDa, disulfide-linked, non-glycosylated homodimer TGF-β1 is cleaved from the C-terminus of a disulfide-linked dimer of pro-TGF-β1 by a subtilisin-like pro-protein convertase protease. It is normally secreted as an inactive, or latent, complex. There are two types of latent complexes: (1) a small latent complex that contains TGF-β1 noncovalently bound to a disulfide-linked dimer of the N-terminal part of pro-TGF-β1, referred to as latency associated peptide (LAP); and (2) a large latent complex that also contains latent TGF-β1 binding protein (LTBP) disulfide-linked to LAP. LTBP may facilitate secretion or targeting of latent TGF-β. The latency proteins also contribute stability. Free TGF-β has a half life of about two minutes, whereas latent TGF-β has a half-life of about 90 minutes. Biological activity requires release of TGF-β1 from the latent complex. This can be done in vitro by disruption of LAP (e.g., via acidification). The physiological mechanism of release from latency, an important control for the regulation and localization of TGF-β activity may occur though proteolysis of LAP. TGF-β1 is synthesized by virtually all cells, and TGF receptors are expressed by all cells. TGF-β exhibits three fundamental activities: (1) it modulates cell proliferation, typically as a supressor; (2) it enhances the deposition of extracellular matrix through promotion of synthesis and inhibition of degradation; and (3) it may be immunosuppressive.

The observation that TGF-β is synthesized by glomerular podocytes and leads to detectable levels in the urine raises the question of whether urinary TGF-β levels can be used as a biomarker of podocyte viability. In early work by Azar et al., urinary TGF-β levels were significantly higher than normal controls, but the effect of tight glucose control on plasma and urinary levels remain controversial [3]. However, studies suggest that the protective effects of ACE or ARB therapy in patients with diabetic nephropathy are mediated in part by a reduction in urinary TGF-β expression. For example, Houlihan et al. followed 21 patients with type II diabetes and microalbuminuria and examined the effect of Losartan on urinary TGF-β levels. In a randomized, double-blind study; patients received Losartan (50 mg) or placebo for a 4 week period followed by 4 week washout. Total urinary TGF-β levels correlated with metabolic control such that TGF-β levels rose in concert with rising blood glucose. While plasma TGF-β levels were not altered by Losartan, urinary levels were significantly reduced [8]. Similar observations have been shown with ACE inhibitors and thiazolinedione therapy [12; 10].

In one embodiment, a patient receiving effective treatment for diabetic nephropathy with Acthar® gel provides a urinary sample before, during, and/or after treatment. The observation that TGF-β is synthesized by glomerular podocytes and leads to detectable levels in the urine raises the question of whether urinary TGF-β levels can be used as a biomarker of podocyte viability. In early work by Azar et al., urinary TGF-β levels were significantly higher than normal controls, but the effect of tight glucose control on plasma and urinary levels remain controversial [3]. However, studies suggest that the protective effects of ACE or ARB therapy in patients with diabetic nephropathy are mediated in part by a reduction in urinary TGF-β expression. For example, Houlihan et al. followed 21 patients with type II diabetes and microalbuminuria and examined the effect of Losartan on urinary TGF-β levels. In a randomized, double-blind study; patients received Losartan (50 mg) or placebo for a 4 week period followed by 4 week washout. Total urinary TGF-β levels correlated with metabolic control such that TGF-β levels rose in concert with rising blood glucose. While plasma TGF-β levels were not altered by Losartan, urinary levels were significantly reduced [8]. Similar observations have been shown with ACE inhibitors and thiazolinedione therapy [12; 10].

The present inventors examine herein for the first time the effects of pharmacologic doses of Acthar® Gel on urinary TGF-β, MCP-1 and VEGF A and VEGF B levels in patients with diabetes mellitus and nephrotic range proteinuria. Specifically, persistent elevation of TGF-β may lead to reduction in end-organ expression of melanocortin receptors and a downstream loss of VEGF production. Moreover, the resulting up-regulation of inflammatory cytokines such as MCP-1 contributes to interstitial fibrosis and loss of nephron mass over time.

### ACTH

ACTH is a hormone that is secreted by the pituitary gland and is a part of the hypothalamus-pituitary-adrenal (HPA) axis that maintains the stress response and homeostasis in the body. In some instances ACTH plays a role in motor neuron function.

ACTH is a 39 amino acid peptide hormone secreted by the anterior pituitary gland. ACTH is secreted from the anterior pituitary in response to corticotropin-releasing hormone (CRH) that is secreted from the hypothalamus. The release of ACTH stimulates the adrenal cortex with subsequent increased production of glucocorticosteroids and/or cortisol from the adrenal cortex.

ACTH is synthesized from a precursor polypeptide pre-pro-opiomelanocortin (pre-POMC). The removal of the signal peptide during translation produces a 267 amino acid polypeptide POMC. POMC undergoes a series of post-translational modifications to yield various polypeptide fragments including and not limited to ACTH, β-lipotropin, γ-lipotropin, α, β, γ-Melanocyte Stimulating Hormone (MSH) and β-endorphin POMC, ACTH and (3-lipotropin are also secreted from the pituitary gland in response to the hormone corticotropin-releasing hormone (CRH). In some embodiments, the first 13 amino acids of ACTH₁₋₃₉ are cleaved to form α-melanocyte-stimulating hormone (α-MSH).

In some instances, multiple hypothalamic, pituitary, and peripheral factors regulate stress-mediated or inflammation-induced POMC expression and/or ACTH secretion. Essential cellular functions maintaining metabolic and neuroendocrine control require a homeostatic, non-stressed pattern of ACTH and glucocorticoid secretion. ACTH secretion is characterized by both circadian periodicity and ultradian pulsatility that is generated by CRH release and is also influenced by peripheral corticosteroids. Thus, ACTH secretion peaks at about before 7 am and nadir adrenal steroid secretion occurs between about 11 pm and 3 am, with periodic secretory bursts occurring throughout the day. Serum cortisol levels also exhibit a similar pattern of circadian periodicity. These rhythms are further reinforced by visual cues and the light-dark cycle. In some instances, stress results in increased ACTH pulse amplitude

In some instances, an abnormality in ACTH levels is associated with inflammation (e.g., increased release of pro-inflammatory cytokines). In some instances, an abnormality in ACTH levels is associated with reduced VEGF secretion. In some instances, reduced VEGF secretion is associated with reduced growth of new blood vessels and inadequate oxygen supply to tissues (e.g., neurons and/or muscles).

ACTH may promote axonal regeneration, act as a neutrophic factor, or increase muscle action potential. In some instances, ACTH levels in the body increase upon activation of certain glutamate receptors such as the NMDA receptors. In other instances, excitotoxicty increases or decreases secretion of ACTH.

### ACTH Forms

The term “ACTH” includes corticotropin, adrenocorticotropic hormone, Tetracosactide or the like. The term “ACTH” also includes any ACTH peptide or any ACTH preparation as described herein. In some embodiments, ACTH is an ACTH peptide. As used herein, in some embodiments, “ACTH peptide” refers to ACTH₁₋₃₉ peptide of structure:

```
H-Ser-Tyr-Ser-Met-Glu-His-Phe-Arg-Trp-Gly-
        1 2 3 4 5 6 7 8 9 10
Lys-Pro-Val-Gly-Lys-Lys-Arg-Arg-Pro-Val-
  11 12 13 14 15 16 17 18 19 20
Lys-Val-Try-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```

or any homologs, analogs, fragments, complexes or aggregates thereof. The term ACTH includes peptides or peptide fragments, complexes, salts or aggregates with about 40%, about 45%, about 50%, about 55%, about 60%, about 65%, about 70%, about 75%, about 80%, about 85%, about 90%, or about 95% homology with ACTH₁₋₃₉. The term ACTH includes ACTH from any source including human ACTH, mouse ACTH, rat ACTH, porcine ACTH, sheep ACTH, bovine ACTH, rabbit ACTH or any other source of ACTH.

In some embodiments, ACTH is an ACTH preparation. As used herein, “ACTH preparation” refers to a mixture containing ACTH peptide and/or other peptide fragments and/or other proteins and/or other substances that together form a composition that is suitable for any methods and/or dosing regimen described herein. In some of such embodiments, ACTH is obtained from a homogenized pituitary extract of an appropriate animal (e.g., pituitary extract of a pig). Any suitable method is used to obtain a homogenized pituitary extract. In some of such embodiments, a homogenized pituitary extract includes ACTH peptide and/or other peptide fragments and/or other proteins and/or other substances that are contemplated as being part of the ACTH preparation that is compatible with any method described herein.

The term ACTH includes humanized and/or recombinant forms of ACTH and synthetic forms of ACTH. The term ACTH includes fragments of ACTH₁₋₃₉. Examples of synthetic forms and/or fragments of ACTH include and are not limited to ACTH₁₋₂₄ peptide having the formula:

```
H-Ser-Tyr-Ser-Met-Glu-His-Phe-Arg-Trp-Gly-
        1 2 3 4 5 6 7 8 9 10
Lys-Pro-Val-Gly-Lys-Lys-Arg-Arg-Pro-Val-
  11 12 13 14 15 16 17 18 19 20
Lys-Val-Try-Pro
  21 22 23 24
```

or a fragment, complex, aggregate, or analog thereof, or any combination thereof,

ACTH₁₋₁₇ peptide having the formula:

```
H-Ser-Tyr-Ser-Met-Glu-His-Phe-Arg-Trp-Gly-
        1 2 3 4 5 6 7 8 9 10
Lys-Pro-Val-Gly-Lys-Lys-Arg-
  11 12 13 14 15 16 17
```

or a fragment, complex, aggregate, or analog thereof, or any combination thereof,

ACTH₄₋₁₀ peptide (ORG-066) of formula:

Met-Glu-His-Phe-Arg-Trp-Gly
4 5 6 7 8 9 10

or a fragment, complex, aggregate, or analog thereof, or any combination thereof, or

ACTH₄₋₉ peptide analog (ORG-2766) of formula:

O₂-Met-Glu-His-Phe-D-Lys-Phe-OH

or a fragment, complex, aggregate, or analog thereof, or any combination thereof.

The term ACTH includes a peptide of formula

Ac-Ser-Tyr-Ser-Met-Glu-His-Phe-Arg-Trp-Gly-Lys-
Pro-Val

or a fragment, complex, aggregate, or analog thereof, or any combination thereof, or a peptide fragment of formula:

H-Ser-Tyr-Ser-Met-Glu-His-Phe-Arg-Trp-Gly-Lys-
Pro-Val

or a fragment, complex, aggregate, or analog thereof, or any combination thereof, or a peptide fragment of formula:

D-Ala-Gln-Tyr-Phe-Arg-Trp-Gly-NH₂.

or a fragment, complex, aggregate, or analog thereof, or any combination thereof.

The term ACTH also includes synthetic preparations of ACTH that are commercially available including and not limited to ACTHAR® powder for injection or gel, Synacthen®, Adrenomone®, or the like. Examples of commercially available ACTH peptides that are compatible with the methods described herein include and are not limited to Adrenocorticotropic Hormone (ACTH) (1-10) (human), Adrenocorticotropic Hormone (ACTH) (1-13) (human), Adrenocorticotropic Hormone (ACTH) (1-16) (human), Adrenocorticotropic Hormone (ACTH) (1-17) (human), Adrenocorticotropic Hormone (ACTH) (1-24) (human), Adrenocorticotropic Hormone (ACTH) (1-39) (human), Adrenocorticotropic Hormone (ACTH) (1-39) (rat), Adrenocorticotropic Hormone (ACTH) (18-39) (human), Adrenocorticotropic Hormone (ACTH) (4-10) (human), Adrenocorticotropic Hormone (ACTH) (1-4), Adrenocorticotropic Hormone (ACTH) (1-14) or the like available from, for example, GenScript.

As used herein, the term ACTH also includes pre-POMC, POMC, β-lipotropin, γ-lipotropin, Melanocyte Stimulating Hormone (α-MSH, β-MSH, γ-MSH), β-endorphin, or the like, or any other polypeptide fragment that is a post-translational product of the POMC gene. POMC genes for various species are found in the NCBI GenBank including and not limited to human POMC transcript variant 1, mRNA, (NCBI Accession number NM_{—}001035256), human POMC transcript variant 2, mRNA, (NCBI Accession number NM_{—}000939), swine pro-opiomelanocortin, mRNA (NCI Accession number S73519), swine proopiomelanocortin protein (POMC) gene (NCBI Accession number EU184858), rat proopiomelanocortin (POMC) gene (NCBI Accession number K01877), or the like. Other examples of POMC genes include, for example, catfish POMC gene described in Animal Genetics, 2005, 36, 160-190.

The term “ACTH analog” or “analog of ACTH” refers to any compounds in which one or more atoms, functional groups, or substructures or amino acids in ACTH or fragments of ACTH have been replaced with different atoms, groups, or substructures or amino acids while retaining the functional activity of the ACTH or fragments of ACTH. In some embodiments, an ACTH analog is a peptide segment of ACTH₁₋₃₉ peptide that retains biological activity of ACTH.

The term “ACTH complex” refers to ACTH or fragments or analogs thereof that are optionally complexed with other proteins (e.g., Bovine Serum Albumin) or metal ions, or fragments of ACTH, or any other suitable complexes that retain the functional characteristics of ACTH or ACTH fragments or analogs thereof and/or allow for formulation of ACTH or ACTH fragments or analogs thereof into suitable dosage forms.

The term “prodrug” refers to a precursor molecule that is a derivative of ACTH or ACTH fragments or analogs thereof that is suitable for incorporation in any dosage form described herein. A “prodrug” refers to a precursor compound that is converted into active compound in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, prodrugs facilitate transmittal across a cell membrane where water solubility is detrimental to mobility but which then is metabolically hydrolyzed to the carboxylic acid, the active entity, once inside the cell where water-solubility is beneficial. As non-limiting examples, a prodrug of ACTH or fragment of analog thereof is metabolically stable and is not degraded in the stomach.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or less labile and/or confers solubility or some other property to the drug. Once the chemical group has been cleaved and/or modified from the prodrug the active drug is generated. In some embodiments, a prodrug of ACTH or fragment or analog thereof is an alkyl ester of the parent compound such as, for example, methyl ester, ethyl ester, n-propyl ester, iso-propyl ester, n-butyl ester, sec-butyl ester, tert-butyl ester or any other ester.

### Methods

ACTH and Regulation of Glomerular Permeability in Diabetic Nephropathy:

In work which preceded the development of oral preparations of glucocorticoids, Farnsworth et al. demonstrated the efficacy of ACTH therapy in children with idiopathic nephrotic syndrome. In subsequent years, the rational for using of ACTH as opposed to glucorticoids is that the trophic effects of ACTH would prevent adrenal cortical atrophy and thereby reduce the risk for an addisonian crisis. However, the observation that melanocortin receptors are expressed in lymphocytes and numerous other cell systems raises the possibility that ACTH receptors may have functions that extend beyond their regulation of glucorticoids. For example, Andersen et al. used in situ hybridization and immunohistochemistry to demonstrate that except for MCR-4, all forms of the melanocortin receptors are expressed on circulating lymphocytes. Interestingly, expression of the ACTH receptor (MCR-2) was greatest in CD4+T cells and to lesser extent CD14+monocytes [2]. The observation that ACTH receptor (MC2R) expression was appreciably higher in CD14 [symbol] ) monocytes than that in granulocytes suggests that ACTH may have a role in the regulation of both active and passive immune pathways. Indeed, stimulation of melanocortin receptors in CD4+ T cells stimulates hapten-specific tolerance and regulates the synthesis of TGF-β[9].

A growing body of evidence suggests a unique interdependence between TGF-β expression and ACTH signaling. For example, Rainey et al. examined the effect of TGF-β on expression of melanocortin receptors in cultured adrenocortical cells. In vitro studies demonstrated that TGF-β significantly decreased melanocortin receptors in primary cultures of adrenal cells. Moreover, TGF-β was able to block the autocrine stimulation by ACTH of its own receptors. The observations suggest that end-organ sensitivity to ACTH could be blunted under conditions where TGF-β levels are chronically elevated. While the precise role of ACTH in podocyte function is unknown, its ability to reduce glomerular permeability in membranous glomerulonephritis suggests that it may regulate slit pore function or serve as a trophic factor for the podocyte itself. Indeed the observation that ACTH can reduce proteinuria in patients with diabetic nephropathy would be consistent with a general effect of ACTH on podocyte function as opposed to reducing proteinuria via its anti-inflammatory properties [4]. Lastly, there may be other links between the overproduction of TGF-β and reduced ACTH responsiveness in patients with diabetic nephropathy. For example, Kechenbauer et al. demonstrated that fibronectin, laminin and type I collagen can reduce ACTH production at the genetic and protein levels [11]. Because TGF-β production in the kidney is increased under hyperglycemic conditions, the enhanced production of extracellular matrix proteins including fibronectin and laminin could reduce local production of ACTH.

Progressive changes in glomerular permeability may involve a complex interaction between TGF-β, loss of functional ACTH receptors and reduced production of vascular endothelial growth factor VEGF. There are three major isoforms of VEGF (121, 165, and 184 which are all a part of the platelet derived growth factor (PDGF) super family. VEGF-165 (VEGF B) has been identified as a splice variant of VEGF 121 (VEGF A). The biologic function of VEGF 165 appears to counteract the effects of VEGF A in that it reduces glomerular permeability and decreases the density of endothelial fenestrations [19]. In animals models of diabetes, the over expression of VEGF 165 leads to reduced proteinuria. In the kidney, VEGF production occurs in the podocyte and the proximal tubule cell [20] where both sources are thought to maintain glomerular and peri-tubular capillary beds. Recent animal models suggest that persistent TGF-β expression leads to a down regulation of ACTH receptor leading to a complicated cascade that ultimately leads to a reduction in functional VEGF type II [21]. As noted by Sugimoto et al., this loss of glomerular VEGF rapidly leads to hypertrophy and damage to glomerular capillaries. These observations, coupled with the deleterious effects of TGF-β on podocyte function, add to the progressive increase in glomerular permeability [25].

### Methods of Monitoring

Provided herein is the utilization of the combination of urinary VEGF 121, VEGF 165 and MCP-1 in monitoring ACTH (e.g., Acthar® Gel) treatment of patients with diabetic nephropathy and nephrotic range proteinuria. As stated above, the term “ACTH” used throughout the methods described herein refers to not only ACTH(1-39), but also, fragments thereof, and analogs thereof, as described in more detail above.

Such methods include monitoring treatment patients having diabetes mellitus or nephrotic range proteinuria, where the patients have been treated with Acthar® Gel. Levels of TGF-β, MCP-1 and VEGF A (VEGF 121) and VEGF B (VEGF 165) may be measured in urine of patients before and after treatment. The present inventors have identified for the first time that the identification of the concentration of these three proteins in combination provides a more accurate diagnosis of a patient needing treatment, a patient which needs increased dosage and/or duration of treatment, a patient needing a reduced dosage and/or duration of treatment, and a patient not needing any additional treatment. Thus, the patients receive more timely and accurate therapy.

Therefore, urinary VEGF 121, VEGF 161, and MCP-1 together may serve as biomarkers of restored glomerular function in patients with diabetic nephropathy and as a clinical means for determining when additional ACTH therapy is needed in patients responsive to ACTH therapy.

The present inventors identified that levels of urinary VEGF 121, VEGF 165 and MCP-1 can function as biomarkers of reduced glomerular permeability in patients being treated with repository corticotropin injections of ACTH for nephrotic range proteinuria secondary to diabetic nephropathy.

Provided herein is a method of identifying a patient for treatment with ACTH, and/or managing the dose and/or duration of ACTH treatment in the long-term management of diabetic nephropathy or nephrotic range proteinuria in a patient comprising: measuring levels of urinary VEGF 121, VEGF 165 and MCP-1 in one or more urine samples obtained from said patient; and adjusting the dose and/or duration of ACTH administered to said patient based on the levels of said VEGF 121, VEGF 165 and MCP-1. ACTH may be administered, for example, as Acthar® gel.

When the ratio of VEGF 121 to VEGF 165 is below about 0.80, a patient begins treatment, or the dose is increased or the duration of treatment is increased. In one embodiment, the ratio is below about 0.75, below about 0.70, below about 0.65, below about 0.60, below about 0.55, or below about 0.50. In another embodiment, the ratio is below 0.80, below 0.75, below 0.70, below 0.65, below 0.60, below 0.55, or below 0.50.

A patient begins treatment, or alternatively, the dose and/or duration of treatment of a patient may be increased if the concentration of urinary VEGF 121 is greater than about 200 pg/mg Cr. In one embodiment, the concentration of urinary VEGF 121 is greater than about 200 pg/mg Cr, about 225 pg/mg Cr, about 250 pg/mg Cr, about 500 pg/mg Cr, about 750 pg/mg Cr, about 1000 pg/mg Cr, about 1250 pg/mg Cr, about 1500 pg/mg Cr, about 1750 pg/mg Cr, about 2000 pg/mg Cr, or more. In another embodiment, the concentration of urinary VEGF 121 is greater than 200 pg/mg Cr, about 225 pg/mg Cr, 250 pg/mg Cr, 500 pg/mg Cr, 750 pg/mg Cr, 1000 pg/mg Cr, 1250 pg/mg Cr, 1500 pg/mg Cr, 1750 pg/mg Cr, 2000 pg/mg Cr, or more.

A patient begins treatment, or alternatively, the dose and/or duration of treatment of a patient may be increased if the concentration of urinary VEGF 165 is greater than about 160 pg/mg Cr. In one embodiment, the concentration of urinary VEGF 165 is greater than about 180 pg/mg Cr, about 200 pg/mg Cr, about 225 pg/mg Cr, about 250 pg/mg Cr, about 500 pg/mg Cr, about 750 pg/mg Cr, about 1000 pg/mg Cr, about 1250 pg/mg Cr, about 1500 pg/mg Cr, about 1750 pg/mg Cr, about 2000 pg/mg Cr, or more. In another embodiment, the concentration of urinary VEGF 165 is greater than 160 pg/ml, 180 pg/mg Cr, 200 pg/mg Cr, 225 pg/mg Cr, 250 pg/mg Cr, 500 pg/mg Cr, 750 pg/mg Cr, 1000 pg/mg Cr, 1250 pg/mg Cr, 1500 pg/mg Cr, 1750 pg/mg Cr, 2000 pg/mg Cr, or more.

A patient begins treatment, or alternatively, the dose and/or duration of treatment of a patient may be increased if the concentration of urinary MCP-1 is greater than about 0.25 ng/mg.

If the concentration of urinary MCP-1 is less than about 0.25 ng/mg, treatment of the patient is stopped, or the dose and/or duration of treatment is decreased.

Alternatively, a patient begins treatment, or alternatively, the dose and/or duration of treatment of a patient may be increased if the concentration of urinary MCP-1 is less than about 1000 mg/24 hours. In one embodiment, the concentration of urinary MCP-1 is less than about 900 mg/24 hours, about 800 mg/24 hours, about 750 mg/24 hours, about 700 mg/24 hours, about 600 mg/24 hours, about 500 mg/24 hours, or less. In another embodiment, the concentration of urinary MCP-1 is less than 900 mg/24 hours, 800 mg/24 hours, 750 mg/24 hours, 700 mg/24 hours, 600 mg/24 hours, 500 mg/24 hours, or less.

Treatment of a patient may cease, or alternatively, the dose and duration of treatment of a patient may be decreased if the concentration of urinary MCP-1 is greater than about 1000 mg/24 hours. In one embodiment, the concentration of urinary MCP-1 is greater than about 1250 mg/24 hours, about 1500 mg/24 hours, about 1750 mg/24 hours, about 2000 mg/24 hours, or more. In another embodiment, the concentration of urinary MCP-1 is greater than 1250 mg/24 hours, 1500 mg/24 hours, 1750 mg/24 hours, 2000 mg/24 hours, or more.

In one aspect of such methods, the concentration of urinary VEGF 121 may be greater than about 200 pg/mg Cr, the concentration of urinary VEGF 165 may be greater than about 160 pg/mg Cr, and the concentration of urinary MCP-1 may be greater than about 0.25 ng/mg, and if the ratio of VEGF 121 to VEGF 165 is below about 0.80, a patient begins treatment, the dose is increased or the duration of treatment is increased.

In another aspect of such methods, the concentration of urinary VEGF 121 may be less than about 200 pg/mg Cr, the concentration of urinary VEGF 165 may be less than about 160 pg/mg Cr, the concentration of urinary MCP-1 may be less than about 0.25 ng/mg, and if the ratio of VEGF 121 to VEGF 165 is above about 0.80, a patient ceases treatment, the dose is decreased or the duration of treatment is decreased.

In another aspect, each dose of ACTH may be increased if a patient is identified as needing further treatment. In one embodiment, a dose concentration of ACTH is increased by about 1 unit, about 2 units, about 3 units, about 4 units, about 5 units, about 6 units, about 7 units, about 8 units, about 9 units, about 10 units, about 12 units, about 14 units or 16 units compared to existing treatment doses of the patient. In one non-limiting example, a dose concentration of 16 units of ACTH may be increased to about 18 units, about 20 units, about 22 units, about 24 units, about 26 units, or more, where one or more doses are administered to a patient undergoing treatment.

In another embodiment, a dose concentration of 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg may be increased by about 10 mg/kg, where one or more doses are administered to a patient undergoing treatment. In yet another embodiment, each dose of ACTH administered to said patient may be increased by about 5%, by about 10%, by about 15%, by about 20%, by about 25%, by about 30%, by about 40%, by about 50% or more.

Where a patient is identified for increased treatment, the frequency of ACTH administration may be increased until the patient responds. For example, a treatment regimen of daily administration of ACTH for 6 months may extended to about 7 months, about 8 months, about 9 months, about 10 months, about 11 months, about 12 months, about 13 months, about 14 months, about 15 months, about 16 months, about 17 months, about 18 months, about 2 years, about 3 years, about 4 years, about 5 years, or more.

Where a patient is receiving a once-daily dose of ACTH, increased treatment may include a twice-daily administration of ACTH.

One would understand that intervals of treatment for patients in remission may be altered as needed if the patient is identified as needing further treatment using a method described herein. For example, in one instance in which a patient in remission was receiving a dose once per month would start receiving a dose every one or two weeks, or depending upon the levels of biomarkers, return to a once daily treatment.

The dosage of ACTH treatment may be decreased if a patient is identified as successfully responding to treatment. In one embodiment, a dose concentration of ACTH may be decreased by about 1 unit, about 2 units, about 3 units, about 4 units, about 5 units, about 6 units, about 7 units, about 8 units, about 9 units, about 10 units, about 12 units, about 14 units or 16 units compared to existing treatment doses of the patient. For example, a dose concentration of 16 units of ACTH may be decreased to about 14 units, about 12 units, about 10 units, about 8 units, or less.

In another embodiment, a dose concentration of 10 mg/kg, 20 mg/kg, 30 mg/kg, 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg or about 200 mg/kg may be decreased by about 5-10 mg/kg. For example, a dose concentration of 150 mg/kg may be decreased to about 145 mg/kg or 140 mg/kg. Alternatively, the dose of ACTH administered to said patient may be reduced by about 5%, about 10% by about 15%, by about 20%, by about 25%, by about 30%, by about 40%, by about 50% or more.

ACTH treatment may be decreased if a patient is identified as successfully responding to treatment. In one embodiment, the duration of ACTH administration is reduced from once every day to once every two days, every three days, every 4 days, every 5 days, every 6 days, every 7 days, every 1.5 weeks, every 2 weeks, every 3 weeks, every 4 weeks, once every 2 months, once every 4 months, once every 6 months or once per year. Additional doses and treatment regimens are described herein below.

One would understand that treatment may be modified in any number of ways: by dose, frequency, intervals of treatment, or a combination thereof. For example, in one instance, a patient having received treatment with ACTH may receive daily treatment for 6-12 months and, thereafter, receive a once-weekly or a once-monthly dose for one, two, three, four, five or more years. In another example, e a once daily or twice daily dose regimen is increased to once per week, once every two weeks, once per month, once every two months, once every three months, once every 6 months or more. Treatment can be modified based upon the methods of monitoring and levels of biomarkersdescribed herein.

In certain specific embodiments of any of the methods described above, the adrenocorticotropic hormone (ACTH) peptide or fragment, analog, complex or aggregate thereof, or any combination thereof, suitable for the methods is ACTH1-39. In certain specific embodiments of any of the methods described above, the adrenocorticotropic hormone (ACTH) peptide or fragment, analog, complex or aggregate thereof, or any combination thereof, suitable for the methods described herein is an ACTH preparation (e.g., ACTHAR®).

Urine samples may be obtained to determine a baseline level of proteins, prior to treatment, at one or more times during treatment, and one or more times post-treatment. Protein levels may be compared to a standard concentration such as described above, to levels of proteins from healthy patients and/or to levels of proteins from sick patients.

One or more additional proteins may be measuring and the results thereof utilized to determine whether or not a patient needs to start treatment, increase the dose and/or duration of treatment, stop treatment, or decrease the dose and/or duration of treatment. Non-limiting examples of proteins to be tested include, but are not limited to, urinary TGF-β, urinary creatine, or both. Proteinuria may also be measured and used in the assessments.

Provided herein is a method of decreasing glomerular permeability in a patient, comprising measuring levels of urinary VEGF 121, VEGF 165 and MCP-1 in one or more urine samples obtained from said patient, wherein if the concentration of urinary VEGF 121 is greater than about 200 pg/mg Cr, the concentration of urinary VEGF 165 is greater than about 160 pg/mg Cr; and the concentration of urinary MCP-1 is greater than about 0.25 ng/mg or less than about 1000 mg/24 hours, the dose and/or duration of ACTH administered to said patient is increased.

In one embodiment, rising levels (concentrations) of VEGF 121 and MCP-1 and decreasing levels of VEGF 165 as biomarkers indicate increasing glomerular permeability, and the dose and/or duration of ACTH treatment is increased.

In another embodiment, decreasing levels of VEGF 121 and MCP-1 and increasing levels of VEGF 165 as biomarkers indicate decreasing glomerular permeability, and the dose and/or duration of ACTH treatment is decreased.

Provided herein is a method of decreasing proteinuria in a patient, comprising measuring levels of urinary VEGF 121, VEGF 165 and MCP-1 in one or more urine samples obtained from said patient, wherein if the ratio of VEGF 121 to VEGF 165 falls below 0.75, proteinuria is diagnosed as increasing, and the dose and/or duration of ACTH administered to said patient is increased.

Provided herein is a method of identifying a patient for treatment with ACTH, and/or increasing the dose and/or duration of ACTH treatment in the long-term management of diabetic nephropathy or nephrotic range proteinuria in a patient comprising measuring levels of urinary VEGF 121 of between about 200 pg/ml and 800 pg/ml, urinary levels of VEGF 165 of between about 180 pg/ml and 2000 pg/ml, and urinary levels of MCP-1 of between about 0.25 ng/mg or 400 mg/24 hours to about 0.4 ng/mg or 1000 mg/24 hours in one or more samples obtained from said patient, identifying a ratio of VEGF 121 to VEGF 165 of below about 0.80, and beginning treatment of said patient or increasing the dose and/or duration of ACTH administered to said patient based on the levels of said VEGF 121, VEGF 165 and MCP-1.

Provided herein is a method of identifying a patient for treatment with ACTH, and/or increasing the dose and/or duration of ACTH treatment in the long-term management of diabetic nephropathy or nephrotic range proteinuria in a patient comprising identifying levels of urinary VEGF 121 of least 200 pg/ml, urinary levels of VEGF 165 of at least 180 pg/ml and urinary levels of MCP-1 of less than about 1000 mg/24 hours or 0.25 ng/mg in one or more samples obtained from said patient and beginning treatment of said patient or increasing the dose and/or duration of ACTH administered to said patient based on the levels of said VEGF 121, VEGF 165 and MCP-1.

Provided herein is a method of identifying a patient for cessation of treatment with ACTH, and/or decreasing the dose and/or duration of ACTH treatment in the long-term management of diabetic nephropathy or nephrotic range proteinuria in a patient comprising measuring levels of urinary VEGF 121 of below about 200 pg/ml, urinary levels of VEGF 165 of below about 180 pg/ml, and urinary levels of MCP-1 of greater than about 1000 mg/24 hours or below about 0.25 ng/mg in one or more samples obtained from said patient; measuring a ratio of VEGF 121 to VEGF 165 of above about 0.80; and ceasing treatment of said patient or decreasing the dose and/or duration of ACTH administered to said patient based on the levels of said VEGF 121, VEGF 165 and MCP-1.

Provided herein is a method of identifying a patient for treatment with ACTH, and/or increasing the dose and/or duration of ACTH treatment in the long-term management of diabetic nephropathy or nephrotic range proteinuria in a subject comprising: measuring levels of urinary VEGF 121, VEGF 165 and MCP-1 in urine samples obtained from said patient, wherein if the ratio of VEGF 121 to VEGF 165 is below about 0.80 and if the level of urinary MCP-1 is less than about 1000 mg/24 hours or above about 0.25 ng/mg, the patient is identified for further treatment, and beginning treatment of said patient or increasing the dose and/or duration of ACTH treatment.

Provided herein is a method of identifying a patient for cessation of treatment with ACTH, and/or decreasing the dose and/or duration of ACTH treatment in the long-term management of diabetic nephropathy or nephrotic range proteinuria in a subject comprising measuring levels of urinary VEGF 121, VEGF 165 and MCP-1 in urine samples obtained from said patient, wherein if the ratio of VEGF 121 to VEGF 165 is above about 0.80 and if the level of urinary MCP-1 is greater than about 1000 mg/24 hours or below about 0.25 ng/mg, the patient is identified as needing reduced treatment; and ceasing treatment of said patient or decreasing the dose and/or duration of ACTH treatment. In one embodiment, the patient is identified as being in remission.

### Methods of Treatment

Provided herein is a method of reducing end-organ expression of melanocortin receptors and downstream loss of VEGF B production in a patient in need thereof, comprising identifying a patient in need of treatment according to the methods described above; and administering ACTH to said patient, wherein downstream loss of VEGF B production is inhibited.

Provided herein is a method of treating patient subject suffering from nephrotic range proteinuria/modulation of glucose levels, comprising identifying a patient in need of treatment according to the methods described above; and administering to said patient one or more doses of an effective amount of ACTH, whereby said patient is treated.

Treatment as described herein includes partial as well as complete (e.g., remission) treatment of a patient. In some cases, treatment includes improvement of one or more symptoms experienced by the patient, reduction in end stage organ failure, reduction in proteinuria, decreased glomerular permeability, reduced podocyte deterioration, or any combination thereof. Improvement can be about 10% or more greater than the state at which the patient was identified for treatment.

Provided herein is a method of inhibiting podocyte failure/degradation in a patient diagnosed with nephrotic range proteinuria comprising identifying a patient in need of treatment according to the methods described above, and administering an effective treatment regimen of ACTH, whereby podocyte failure/degradation in said patient is inhibited.

Provided herein is a method of reducing nephritic range proteinuria in a patient having CKD stage II/III diabetic nephropathy comprising identifying a patient in need of treatment according to the methods described above; and administering one or more doses of an effective amount of ACTH, whereby the concentration of urinary VEGF 165 is increased to at least about 110 pg/ml, and whereby nephritic range proteinuria in a patient is reduced

Provided herein, in some embodiments, are methods of prophylactic treatment comprising administration of ACTH to an individual in need thereof. In some embodiments herein, “prophylactic treatment” refers to effacement of podocyte foot-plate processes and accelerated apoptosis to delay the onset or pathogenesis of diabetic nephropathy. In some embodiments, “prophylactic treatment” refers to treatment before glomerular permeability has been adversely impaired or affected by diabetes mellitus to reduce the likelihood or incidence of diabetes mellitus. In a non-limiting example, a subject at risk for diabetes mellitus can be prophylactically treated according to the present methods prior to undergoing a surgical procedure. In other embodiments, “prophylactic treatment” refers to reducing glomerular permeability such that nephrotic range proteinuria and interstitial fibrosis are reduced. In further embodiments, “prophylactic treatment” refers to treatment after podocyte function has been impaired or affected by diabetes mellitus to reduce the extent of end-organ complications including diabetic nephropathy and nephrotic proteinuria.

In some embodiments, administration of a dosing regimen of adrenocorticotropic hormone (ACTH) peptide or fragment, analog, complex or aggregate thereof, or any combination thereof, as described herein, to an individual suspected of having, predisposed to, or at risk of developing diabetic nephropathy or nephrotic proteinuria maintains urinary protein levels associated and increased with diabetic nephropathy or nephrotic proteinuria (e.g., maintain urinary protein levels without any further changes) in the individual, or changes urinary protein levels to partially normal or substantially normal levels. As used herein, a “change to substantially normal urinary protein levels” refers to a change in physiological levels of urinary protein levels in an individual suspected of having, predisposed to, or at risk of developing a renal disorder to levels that are substantially the same as the levels of urinary protein in a normal individual. As used herein, substantially the same means, for example, about 90% to about 110% of the measured urinary protein levels in a normal individual. In other embodiments, substantially the same means, for example, about 80% to about 120% of the measured urinary protein levels in a normal individual when measured at the about same time. As used herein, “change to partially normal level of urinary protein” refers to any change in urinary protein levels in an individual suspected of having, predisposed to, or at risk of developing diabetic nephropathy or nephrotic proteinuria that trends towards urinary protein levels of a normal individual when measured at about the same time. As used herein “partially normal urinary protein level” is, for example, ±about 25%, ±about 35%, ±about 45%, ±about 55%, ±about 65%, or ±about 75% of the measured urinary protein level of a normal individual when measured at the about same time.

Treatment is considered effective if levels of MCP-1 are reduced post-treatment compared to baseline levels; TGF-β levels in urine are reduced post-treatment compared to baseline levels; levels of VEGF A are reduced post-treatment compared to baseline levels; and levels of VEGF B levels are increased post-treatment compared to baseline levels.

In another embodiment, a patient receiving effective treatment for nephrotic range proteinuria with Acthar® gel provides a urinary sample before, during, and/or after treatment. Treatment is considered effective if levels of MCP-1 are reduced post-treatment compared to baseline levels; TGF-β levels in urine are reduced post-treatment compared to baseline levels; levels of VEGF A are reduced post-treatment compared to baseline levels; and levels of VEGF B levels are increased post-treatment compared to baseline levels.

Also provided herein is a method of assessing podocyte viability by measuring the levels of urinary TGF-β, MCP-1 and VEGF pre- and post-treatment. In one embodiment, podocytes viability is increased post-treatment with Acthar® gel if MCP-1 are reduced post-treatment compared to baseline levels; TGF-β levels in urine are reduced post-treatment compared to baseline levels; levels of VEGF A are reduced post-treatment compared to baseline levels; and levels of VEGF B levels are increased post-treatment compared to baseline levels.

Also provided herein is a method of diagnosing a patient for having diabetic nephropathy for further treatment by measuring urinary levels of TGF-β, MCP-1 and VEGF following cessation of treatment.

Alternatively, a patient having been treated for diabetic nephropathy is identified as being in remission if levels of urinary MCP-1 remain reduced compared to disease levels; TGF-β levels in urine remain reduced post-treatment compared to disease levels; levels of VEGF A remain reduced compared to disease levels; and levels of VEGF B levels remain increased compared to disease levels.

Also provided herein is a method of diagnosing a patient for having nephrotic range proteinuria for further treatment by measuring urinary levels of TGF-β, MCP-1 and VEGF following cessation of treatment.

Alternatively, a patient having been treated for nephrotic range proteinuria is identified as being in remission if levels of urinary MCP-1 remain reduced compared to disease levels; TGF-β levels in urine remain reduced post-treatment compared to disease levels; levels of VEGF A remain reduced compared to disease levels; and levels of VEGF B levels remain increased compared to disease levels.

In some embodiments of the methods of treatment of described herein, the first dose and one or more subsequent doses of ACTH or fragment, analog, complex or aggregate thereof, or any combination thereof, are administered in a dosing regimen that is a pulsed dosing regimen (e.g., the dosing schedule produces escalating ACTH levels early in the dosing interval followed by a prolonged dose-free period). In some embodiments of the methods of treatment of described herein, the first dose and one or more subsequent doses of ACTH or fragment, analog, complex or aggregate thereof, or any combination thereof, are administered in a dosing regimen that is not continuous (i.e., the intervals between doses are uneven). In some embodiments of the methods of treatment of described herein, the first dose and one or more subsequent doses of ACTH or fragment, analog, complex or aggregate thereof, or any combination thereof, are administered in a dosing regimen that is a continuous dosing regimen.

In some embodiments, the first dose is administered upon detection of one or more symptoms of diabetic nephropathy or nephrotic proteinuria. In some embodiments, the first dose is administered upon detection of excess TGFβ or a ratio of VEGF 121 to VEGF 165 of below about 0.80 in urine. In some embodiments, the one or more subsequent doses are administered every day, every other day, every two days, every three days, every four days, every 5 days, every 6 days, once a week, every two weeks, every three weeks, once a month, every six weeks, every two months, every three months, every four months five months, every six months or any combination thereof.

In some embodiments, the dosing regimen comprises doses that produce decreasing levels of drug early in the dosing interval followed by a prolonged dose-free interval. In some embodiments, the dosing regimen comprises a first dose, a series of subsequent doses, followed by a drug holiday, and then, one or more series of doses that are the same as or different from the first series of doses. By way of example only, in one dosing regimen, methods of treatment of described herein comprise administration of ACTH or fragment, analog, complex or aggregate thereof, or any combination thereof, and comprise a first dose of 80 IU, then a once daily dose of 20 IU for three days, followed by a 40 IU dose every week for a month, followed by a drug holiday for 3 months, and then a second series of doses comprising a first dose of 60 IU, then a once daily dose of 20 IU for three days, followed by a 40 IU dose every week for a month, followed by a drug holiday for 3 months.

In some embodiments, a dosing regimen comprises dosing that produces escalating levels of drug early in the dosing interval followed by a prolonged dose-free period. By way of example only, in one dosing regimen, the methods of treatment of described herein comprise administration of ACTH or fragment, analog, complex or aggregate thereof, or any combination thereof, and comprise a first dose of 20 IU, a second dose of 20 IU in the same week, then 40 IU twice a week, then 40 IU every other month for three months.

In some embodiments, a first dose of ACTH or fragment, analog, complex or aggregate thereof, or any combination thereof, is between about 10 IU, 20 IU, 30 IU, 40 IU, 50 IU, 60 IU, 70 IU, 80 IU to about 50 IU, 60 IU, 70 IU, 80 IU, 90 IU, 100 IU, 110 IU, 120 IU, 130 IU, 140 IU, 150 IU or 200 IU. In some embodiments, a first dose of ACTH or fragment, analog, complex or aggregate thereof, or any combination thereof, is between about 10 IU to about 200 IU, between about 10 IU to about 150 IU, between about 10 IU to about 100 IU, between about 10 IU to about 80 IU, between about 10 IU to about 60 IU, or between about 10 IU to about 40 IU. In some embodiments, a first dose of ACTH or fragment, analog, complex or aggregate thereof, or any combination thereof, is between about 10 IU to about 200 IU, between about 20 IU to about 200 IU, between about 40 IU to about 200 IU, between about 40 IU to about 150 IU, between about 40 IU to about 100 IU, between about 40 IU to about 80 IU, or between about 40 IU to about 60 IU. In some embodiments, a first dose of ACTH or fragment, analog, complex or aggregate thereof, or any combination thereof, is between about 20 IU to about 200 IU, between about 60 IU to about 150 IU, between about 60 IU to about 100 IU, or between about 60 IU to about 80 IU.

In some embodiments, a one or more subsequent dose of ACTH or fragment, analog, complex or aggregate thereof, or any combination thereof, is between about 10 IU, 20 IU, 30 IU, 40 IU, 50 IU, 60 IU, 70 IU, 80 IU to about 50 IU, 60 IU, 70 IU, 80 IU, 90 IU, 100 IU, 110 IU, 120 IU, 130 IU, 140 IU, 150 IU or 200 IU. In some embodiments, a one or more subsequent dose of ACTH or fragment, analog, complex or aggregate thereof, or any combination thereof, is between about 10 IU to about 200 IU, between about 10 IU to about 150 IU, between about 10 IU to about 100 IU, between about 10 IU to about 80 IU, between about 10 IU to about 60 IU, or between about 10 IU to about 40 IU. In some embodiments, a one or more subsequent dose of ACTH or fragment, analog, complex or aggregate thereof, or any combination thereof, is between about 20 IU to about 200 IU, between about 20 IU to about 150 IU, between about 20 IU to about 100 IU, between about 20 IU to about 80 IU, or between about 20 IU to about 60 IU, or between about 20 IU to about 40 IU. In some embodiments, a one or more subsequent dose of ACTH or fragment, analog, complex or aggregate thereof, or any combination thereof, is between about 40 IU to about 200 IU, between about 40 IU to about 150 IU, between about 40 IU to about 100 IU, between about 40 IU to about 80 IU, or between about 40 IU to about 60 IU. In some embodiments, a one or more subsequent dose of ACTH or fragment, analog, complex or aggregate thereof, or any combination thereof, is between about 20 IU to about 200 IU, between about 60 IU to about 150 IU, between about 60 IU to about 100 IU, or between about 60 IU to about 80 IU.

Where the ACTH, or fragment, analog, complex or aggregate thereof, or any combination thereof, is a synthetic preparation (i.e., not naturally occurring), in some embodiments, a first dose of ACTH or fragment, analog, complex or aggregate thereof, or any combination thereof, is between about 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg. In some embodiments, a first dose of ACTH or fragment, analog, complex or aggregate thereof, or any combination thereof, is between about 10 mg/kg to about 200 mg/kg, between about 20 mg/kg to about 200 mg/kg, between about 20 mg/kg to about 150 mg/kg, between about 20 mg/kg to about 100 mg/kg, between about 20 mg/kg to about mg/kg IU, between about mg/kg IU to about mg/kg IU, or between about 20 mg/kg to about 40 mg/kg. In some embodiments, a first dose of ACTH or fragment, analog, complex or aggregate thereof, or any combination thereof, is between about 40 mg/kg to about 200 mg/kg, between about 40 mg/kg to about 150 v, between about 40 mg/kg to about 100 mg/kg, between about 40 mg/kg to about 80 mg/kg, or between about 40 mg/kg to about 60 mg/kg. In some embodiments, a first dose of ACTH or fragment, analog, complex or aggregate thereof, or any combination thereof, is between about 20 mg/kg to about 200 mg/kg, between about 60 mg/kg to about 150 mg/kg, between about 60 mg/kg to about 100 mg/kg, or between about 60 mg/kg to about 80 mg/kg.

Where the ACTH, or fragment, analog, complex or aggregate thereof, or any combination thereof, is a synthetic preparation (i.e., not naturally occurring), in some embodiments, a one or more subsequent dose of ACTH or fragment, analog, complex or aggregate thereof, or any combination thereof, is between about 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg. In some embodiments, a one or more subsequent dose of ACTH or fragment, analog, complex or aggregate thereof, or any combination thereof, is between about 10 mg/kg to about 200 mg/kg, between about 10 mg/kg to about 150 mg/kg, between about 10 mg/kg to about 100 mg/kg, between about 10 mg/kg to about 80 mg/kg IU, between about 10 mg/kg IU to about 60 mg/kg IU, or between about 10 mg/kg to about 40 mg/kg. In some embodiments, a one or more subsequent dose of ACTH or fragment, analog, complex or aggregate thereof, or any combination thereof, is between about 20 mg/kg to about 200 mg/kg, between about 20 mg/kg to about 150 mg/kg, between about 20 mg/kg to about 100 mg/kg, between about 20 mg/kg to about 80 mg/kg, or between about 20 mg/kg to about 60 mg/kg. In some embodiments, a one or more subsequent dose of ACTH or fragment, analog, complex or aggregate thereof, or any combination thereof, is between about 40 mg/kg to about 200 mg/kg, between about 40 mg/kg to about 150 mg/kg, between about 40 mg/kg to about 100 mg/kg, between about 40 mg/kg to about 80 mg/kg, or between about 40 mg/kg to about 60 mg/kg. In some embodiments a one or more subsequent dose of ACTH or fragment, analog, complex or aggregate thereof, or any combination thereof, is between about 20 mg/kg to about 200 mg/kg, between about 60 mg/kg to about 150 mg/kg, between about 60 mg/kg to about 100 mg/kg, or between about 60 mg/kg to about 80 mg/kg.

In some embodiments, a one or more subsequent dose of ACTH or fragment, analog, complex or aggregate thereof, or any combination thereof, is between about 10%-90%, between about 20%-80%, between about 20%-60%, or between about 20%-40% of the first dose of ACTH or fragment, analog, complex or aggregate thereof, or any combination thereof. In some embodiments, a one or more subsequent dose of ACTH or fragment, analog, complex or aggregate thereof, or any combination thereof, is between about 80%-200%, between about 80%-175%, between about 80%-150%, between about 80%-125%, or between about 80%-100% of the first dose of ACTH or fragment, analog, complex or aggregate thereof, or any combination thereof.

In some embodiments, where the patient's condition does not improve upon administration of a dosing regimen as identified by a method described above, upon the doctor's discretion the ACTH peptide or fragment, analog, complex or aggregate thereof, or any combination thereof is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the ACTH peptide or fragment, analog, complex or aggregate thereof, or any combination thereof is optionally given continuously; alternatively, the dose of ACTH peptide or fragment, analog, complex or aggregate thereof, or any combination thereof being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose may be administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of ACTH peptide or fragment, analog, complex or aggregate thereof, or any combination thereof. In some embodiments, the unit dosage is in the form of a package containing discrete quantities of the formulation. Non-limiting examples are packaged tablets or capsules, powders in vials or ampoules, or injectable suspension or solution in ampoules. In some embodiments, aqueous suspension compositions are packaged in single-dose non-reclosable containers. Alternatively, multiple-dose reclosable containers are used. In some of such embodiments, a preservative is optionally included in the composition. By way of example only, formulations for intramuscular injection are presented in unit dosage form, which include, but are not limited to ampoules, or in multi dose containers, with an added preservative.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. ACTH peptide or fragment, analog, complex or aggregate thereof, or any combination thereof, exhibiting high therapeutic indices are preferred. The dosage of such ACTH peptide or fragment, analog, complex or aggregate thereof, or any combination thereof lies preferably within a range of circulating concentrations that include the ED50 with minimal toxicity. The dosage optionally varies within this range depending upon the dosage form employed and the route of administration utilized.

Provided herein, in certain embodiments, are compositions comprising at least one ACTH peptide or fragment, analog, complex or aggregate thereof, or any combination thereof, where the ACTH peptide or fragment, analog, complex or aggregate thereof, or any combination thereof, is as described herein.

Pharmaceutical compositions are formulated using one or more physiologically acceptable carriers including excipients and auxiliaries which facilitate processing of the ACTH peptide or fragment, analog, complex or aggregate thereof, or any combination thereof, into preparations which are used pharmaceutically. Proper formulation is dependent upon the route of administration chosen. A summary of pharmaceutical compositions is found, for example, in Remington: The Science and Practice of Pharmacy, Nineteenth Ed (Ea hston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more of ACTH peptide or fragment, analog, complex or aggregate thereof, or any combination thereof, and a pharmaceutically acceptable diluent(s), excipient(s), or carrier(s). In addition, the ACTH peptide or fragment, analog, complex or aggregate thereof, or any combination thereof, is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of ACTH peptide or fragment, analog, complex or aggregate thereof, or any combination thereof, with other chemical components, such as carriers, stabilizers, diluents, dispersing agents, suspending agents, thickening agents, and/or excipients. In some embodiments, a pharmaceutical composition comprises an ACTH preparation (e.g., an ACTH peptide or fragment, analog, complex or aggregate thereof, or any combination thereof, and any other proteins and/or other substances that are present in a homogenized pituitary extract obtained from an appropriate animal source) and other chemical components, such as carriers, stabilizers, diluents, dispersing agents, suspending agents, thickening agents, and/or excipients. The pharmaceutical composition facilitates administration of ACTH peptide or fragment, analog, complex or aggregate thereof, or any combination thereof, to an organism. In practicing the methods of treatment or use provided herein, an ACTH peptide or fragment, analog, complex or aggregate thereof, or any combination thereof, are administered in a pharmaceutical composition to a mammal having a condition, disease, or disorder to be treated. Preferably, the mammal is a human. The does and dosing regimen varies depending on the severity and stage of the condition, the age and relative health of an individual, the potency of ACTH peptide or fragment, analog, complex or aggregate thereof, or any combination thereof, used and other factors. The ACTH peptide or fragment, analog, complex or aggregate thereof, or any combination thereof, is optionally used singly or in combination with one or more therapeutic agents as components of mixtures.

The pharmaceutical formulations described herein are optionally administered to a individual by multiple administration routes, including but not limited to, oral, parenteral (e.g., intravenous, subcutaneous, intramuscular, intrathecal), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one ACTH peptide or fragment, analog, complex or aggregate thereof, or any combination thereof, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of ACTH peptide or fragment, analog, complex or aggregate thereof, or any combination thereof, having the same type of activity. In some situations, ACTH peptide or fragment, analog, complex or aggregate thereof, or any combination thereof, exist as tautomers and/or rotational isomers. All tautomers and/or rotational isomers are included within the scope of the embodiments presented herein. Additionally, ACTH peptide or fragment, analog, complex or aggregate thereof, or any combination thereof, exists in unsolvated as well as solvated forms with pharmaceutically acceptable solvents such as water, ethanol, and the like. The solvated forms of the ACTH peptide or fragment, analog, complex or aggregate thereof, or any combination thereof, presented herein are also considered to be disclosed herein. In some embodiments, ACTH peptide or fragment, analog, complex or aggregate thereof, or any combination thereof, exists as a complex with metal ions. The metal-ion complexed forms of the ACTH peptide or fragment, analog, complex or aggregate thereof, or any combination thereof, presented herein are also considered to be disclosed herein.

“Carrier materials” include any commonly used excipients in pharmaceutics and should be selected on the basis of compatibility with ACTH peptide or fragment, analog, complex or aggregate thereof, or any combination thereof, disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a ACTH peptide or fragment, analog, complex or aggregate thereof, or any combination thereof, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising a ACTH peptide or fragment, analog, complex or aggregate thereof, or any combination thereof, is a solid drug dispersion. A solid dispersion is a dispersion of one or more active ingredients in an inert carrier or matrix at solid state prepared by the melting (or fusion), solvent, or melting-solvent methods. (Chiou and Riegelman, Journal of Pharmaceutical Sciences, 60: 1281 (1971)). The dispersion of one or more active agents in a solid diluent is achieved without mechanical mixing. Solid dispersions are also called solid-state dispersions. In some embodiments, any ACTH peptide or fragment, analog, complex or aggregate thereof, or any combination thereof, described is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into powders for reconstitution. Solubility of an SDD comprising a drug is higher than the solubility of a crystalline form of a drug or a non-SDD amorphous form of a drug. In some embodiments of the methods described herein, ACTH peptide or fragment, analog, complex or aggregate thereof, or any combination thereof, are administered as SDDs constituted into appropriate dosage forms described herein.

Pharmaceutical preparations for oral use are optionally obtained by mixing one or more solid excipient with a ACTH peptide or fragment, analog, complex or aggregate thereof, or any combination thereof, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, a prodrug of the ACTH peptide or fragment, analog, complex or aggregate thereof, or any combination thereof, is used in preparations for oral use.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of an ACTH peptide or fragment, analog, complex or aggregate thereof, or any combination thereof, are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a ACTH peptide or fragment, analog, complex or aggregate thereof, or any combination thereof, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a ACTH peptide or fragment, analog, complex or aggregate thereof, or any combination thereof, are optionally further formulated to provide a controlled release of the ACTH peptide or fragment, analog, complex or aggregate thereof, or any combination thereof. Controlled release refers to the release of the ACTH peptide or fragment, analog, complex or aggregate thereof, or any combination thereof, from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a individual over an extended period of time according to a predetermined profile. Such release rates provide levels of ACTH peptide or fragment, analog, complex or aggregate thereof, or any combination thereof, for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a ACTH peptide or fragment, analog, complex or aggregate thereof, or any combination thereof, are delivered using a pulsatile dosage form. A pulsatile dosage form is capable of providing one or more immediate release pulses at predetermined time points after a controlled lag time or at specific sites. Pulsatile dosage forms including the formulations described herein, which include a ACTH peptide or fragment, analog, complex or aggregate thereof, or any combination thereof, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the ACTH peptide or fragment, analog, complex or aggregate thereof, or any combination thereof, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the ACTH peptide or fragment, analog, complex or aggregate thereof, or any combination thereof, is optionally in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the ACTH peptide or fragment, analog, complex or aggregate thereof, or any combination thereof, and a suitable powder base such as lactose or starch.

Buccal formulations that include a ACTH peptide or fragment, analog, complex or aggregate thereof, or any combination thereof, include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the ACTH peptide or fragment, analog, complex or aggregate thereof, or any combination thereof, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of an ACTH peptide or fragment, analog, complex or aggregate thereof, or any combination thereof, are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of a ACTH peptide or fragment, analog, complex or aggregate thereof, or any combination thereof; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a ACTH peptide or fragment, analog, complex or aggregate thereof, or any combination thereof, employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the ACTH peptide or fragment, analog, complex or aggregate thereof, or any combination thereof, is optionally accomplished by means of iontophoretic patches and the like. Additionally, transdermal patches provide controlled delivery of the ACTH peptide or fragment, analog, complex or aggregate thereof, or any combination thereof. The rate of absorption is optionally slowed by using rate-controlling membranes or by trapping the ACTH peptide or fragment, analog, complex or aggregate thereof, or any combination thereof, within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the ACTH peptide or fragment, analog, complex or aggregate thereof, or any combination thereof, optionally with carriers, optionally a rate controlling barrier to deliver the ACTH peptide or fragment, analog, complex or aggregate thereof, or any combination thereof, to the skin of the host at a controlled and predetermined rate over a prolonged period of time, and means to secure the device to the skin.

Formulations that include a ACTH peptide or fragment, analog, complex or aggregate thereof, or any combination thereof, suitable for intramuscular, intrathecal, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a ACTH peptide or fragment, analog, complex or aggregate thereof, or any combination thereof, is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections including intrathecal and intramuscular injections, appropriate formulations include aqueous or non-aqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the ACTH peptide or fragment, analog, complex or aggregate thereof, or any combination thereof, in water soluble form. Additionally, suspensions of the ACTH peptide or fragment, analog, complex or aggregate thereof, or any combination thereof, are optionally prepared as appropriate oily injection suspensions.

In some embodiments, the ACTH peptide or fragment, analog, complex or aggregate thereof, or any combination thereof, is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The ACTH peptide or fragment, analog, complex or aggregate thereof, or any combination thereof, is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Systems and, Non-Transitory Computer-Readable Storage Media

Provided herein is a system for identifying a patient for treatment with ACTH(1-39), a fragment thereof, or an analog thereof, and/or managing the dose, interval and/or duration of ACTH treatment in the long-term management of diabetic nephropathy or nephrotic range proteinuria in a patient comprising antibodies that specifically bind to urinary VEGF 121, VEGF 165 and MCP-1; an optical density microplate reader; and a composition comprising ACTH(1-39), a fragment thereof, or an analog thereof. In one embodiment, the system further comprises an optionally networked computer processing device configured to perform executable instructions; and a computer program, the computer program comprising a software module executed by the computer processing device to apply a model or algorithm for analyzing the urinary levels of VEGF 121, VEGF 165 and MCP-1 in the sample.

Provided herein is a computer-implemented system comprising: (a) a computer comprising: a processor, an operating system configured to perform executable instructions, and a memory device; and (b) a computer program including instructions executable by the computer, the program comprising (i) a software module configured to receive data indicating levels of urinary VEGF 121, VEGF 165 and MCP-1 in one or more urine samples obtained from a patient, the patient in need of long-term management of diabetic nephropathy or nephrotic range proteinuria; (ii) a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and (iii) a software module configured to generate a report comprising a recommendation for an adjustment to the dose, interval and/or duration of ACTH(1-39), a fragment thereof, or an analog thereof administered to the patient.

In one embodiment, the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

In another embodiment, the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.

Also provided herein is a non-transitory computer-readable storage media encoded with a computer program including instructions executable by a processor, the program comprising (a) a software module configured to receive data indicating levels of urinary VEGF 121, VEGF 165 and MCP-1 in one or more urine samples obtained from a patient, the patient in need of long-term management of diabetic nephropathy or nephrotic range proteinuria; (b) a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and (c) a software module configured to generate a report comprising a recommendation for an adjustment to the dose, interval and/or duration of ACTH(1-39), a fragment thereof, or an analog thereof administered to the patient.

In one embodiment, the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

In another embodiment, the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.

### Digital Processing Device

In some embodiments, the methods, systems, and software described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

### Non-Transitory Computer Readable Storage Medium

In some embodiments, the methods, systems, and software disclosed herein include one or more computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the methods, systems, and software disclosed herein include at least one computer program, or use of the same. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The methods, systems, and software disclosed herein include, in various embodiments, software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods, systems, and software disclosed herein include one or more databases, or use of the same. In view of the disclosure provided herein, those of skill in the art will recognize that many databases are suitable for storage and retrieval of metagenomic information (including metagenomic profiles), metatranscriptome information (including metatranscriptome profiles), and multiplex profiles. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local storage devices.

In order that those in the art may be better able to practice the compositions and methods described herein, the following examples are provided for illustrative purposes.

### EXAMPLES

### Example 1

### Protocol Design: Clinic Visits:

Screening and Consenting for Protocol Inclusion:

Patients with type I or type II diabetes mellitus requiring medical treatment of hyperglycemia are screened using SERRI CKD database and registry. Patients are contacted by telephone where a preliminary discussion of the protocol is made. Patients expressing an interest in participating in the study are invited to meet with one or more primary investigators and/or clinical coordinator staff with the research group. There is a detailed conversation regarding the risk benefits of participating in the trial. Patients agreeing to participate in the study are asked to give written informed consent.

Inclusion-Exclusion Criteria:

Patients meeting the following inclusion exclusion criteria are considered eligible for study participation.

Inclusion criteria for participation in the study include the following:
a) Age>18 and ≦80;
b) Type I or Type II Diabetes Mellitus;
c) Stable ACE or ARB therapy for 4 weeks prior to study enrollment;
d) Urinary protein>3000 mg/24 hrs; and
e) Patients with more than one protein lowering agent (e.g., ACE or ARB, or MR antagonist or Tekturna require two consecutive 24 hour urinary protein of 2000 mg/24 hrs.

Exclusion criteria for participation in the study include the following:
a) Age<18 or >80;
b) HgbAlc>9.0% or 11% if using the (DCCT/NGSP) method;
c) eGFR<20 mls/min by MDRD formula or eGFR by (Cockoff-Gault 20 mls/min);
d) Dilated cardiomyopathy with known EF<40%;
e) Pregnant or nursing mothers;
f) Patients with an admission for diabetic ketoacidosis, or non-ketotic hyperosmolar coma within 6 months of study enrollment;
g) Patients with known mixed glomerulonephritis and diabetic glomerulopathy;
h) Patients within 3 months of operative procedures or chronic non-healing wounds;
i) Patients with glucocorticoid-induced diabetes mellitus;
j) Patients with known sensitivity to porcine protein products; and
k) Patients with bleeding gastric or duodenal ulcers requiring hospitalization six months prior to study enrollment.

A primary endpoint of the study is: Percentage of patients achieving less than 300 mg protein per 24 hours after 6 months of Acthar® Gel.

One example of a secondary endpoint is: Percentage of patients achieving greater than 50% reduction in urinary proteinuria after 6 months of Acthar® Gel.

Another example of a secondary endpoint is: The percent change in urinary VEGF following 6 months of ACTH therapy.

Safety endpoints include, for example, the percentage of patients achieving one of the following endpoints:
a) Patients with HgBAlc from Days 28, 56, and 84 that averages >8.5% despite a reduction in the dose of Acthar® Gel or increasing doses of insulin and/or oral agents were withdrawn from the study;
b) Patients with 2 or more episodes with measured serum Blood glucose >600 mg/dl despite a reduction in the dose of Acthar® Gel or increasing doses of insulin and/or oral agents will be withdrawn from the study;
c) Patients with 2 or more episodes of documented accelerated hypertension;
d) (BP>210/120) despite confirmed compliance with antihypertensive medications will be withdrawn from the protocol; or
e) Patients with recurrent congestive heart failure despite modifications in diuretic and antihypertensive therapy will be withdrawn from the study.

Days (−28 to −2): Baseline Proteinuria Period:

Patients giving written informed consent receive a complete history and physical (H&P) exam. A careful problem list outlining major milestones in the patient's medical history is created. All patients participating in this protocol have copies of their H&P scanned into their electronic medical record. In addition, their primary MD and principal nephrologist are contacted as to their participation in the study.

Participating patients are asked to collect a 24 hour urine sample for albumin. This is delivered to the SERRI research clinic. Patients at that time have the following blood samples obtained:

1) CBC with differential and platelet count;

2) Complete metabolic panel with PO₄;

3) Plasma HgBAlc;

4) Fasting Lipid Profile; and

5) Serum Aldosterone level.

The patient will also be asked to provide approximately 100 mLs of freshly voided urine for measurement of Urinary TGF-β concentrated. A portion of that urine sample is sent for measurement of Protein/Cr ratio. This sample is used as validation of the 24 hour urine collection. If it is determined that the 24 hour collection is invalid, the protein/Cr ratio is used as the measurement of albuminuria and the Cr in this urine is used to normalize the TGF-β/Cr ratio.

Day 0: Randomization and First Day of Drug Injection:

Laboratory Data:

Patients will be asked to collect a second 24 hour urine sample for albumin. This is delivered to the SERRI research clinic on Day 0 of the study. Patients at that time also have the following blood samples obtained:

1) CBC with differential and platelet count;

2) Complete metabolic panel with PO₄;

3) Plasma HgBAlc;

4) Fasting Lipid Profile; and

5) Serum Aldosterone level.

The two 24 hour urine collections for protein and the two serum Aldosterone levels are averaged to represent the baseline values for both these parameters.

Urinary VEGF 121, VEGF-165, MCP-1 and TGF-β collection:

Patients are asked to provide approximately 100 mls of freshly voided urine on the morning of the Day 0 and then monthly throughout the 6 month study. All samples are stored at −80° C. until assayed.

Acthar® Gel Randomizations:

Using a sealed envelope method, patients giving written informed consent are randomized to 16 units or 32 units of Acthar® Gel subcutaneously (SQ) every day for 6 months.

All patients participating in the study are instructed in the preparation and administration of the Acthar® Gel.

All patients will have the first dose administered in the SERRI clinic where patients are observed for 1 hour to accommodate potential allergic or anaphylactic reactions.

The primary investigator may determined if the randomization dose of Acthar® Gel is to be reduced from 32 units to 16 units for (1) blood glucose>600 mg/dl, (2) total cholesterol<100 mg/dl or (3) greater than a 50% reduction from baseline.

Urinary VEGF-A (isoforms 120, 165, and 184), VEGF-B (165), Monocyte Chemotactic Peptide-1 (MCP-1) and Transforming Growth Factor Beta (Isoforms 1, 2 and 3) are Measured Pre- and Post-ACTH Therapy

Urine samples are obtained from patients as follows: baseline, and at 1 month, at 3 months, and at 6 months.

The effects of the two doses of ACTH (16 & 32 units) on the above-identified urinary proteins is measured by commercially available kits. This will employ both ELISA and Luminex assays and, if needed, the results will be confirmed by western blot analysis. Each patient will have between 5-6 samples. All time points are measured in triplicate in the laboratory of Dr. Brad Rovin, Renal Division, Ohio State University Columbus Ohio.

It is expected that ACTH therapy will reduce urinary TGF-β, while increasing VEGF A and VEGF-B in patients suffering from diabetes mellitus. However, total amount VEGF-A may remain unchanged with the primary protein lowering effect being the result of rising VEGF-B. The rational is that recent data suggests a regulatory role of ACTH on both the expression of the native peptide and its receptor [18].

A positive beneficial effect of ACTH may be mediated through a VEGF dependent restoration of glomerular function.

Results

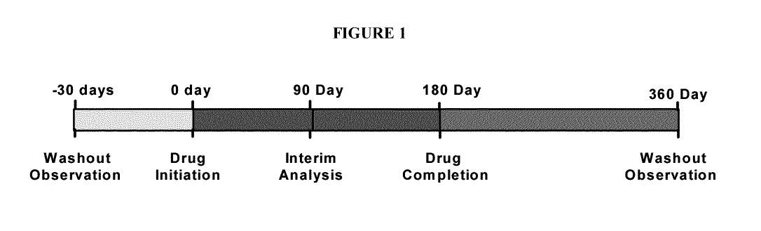

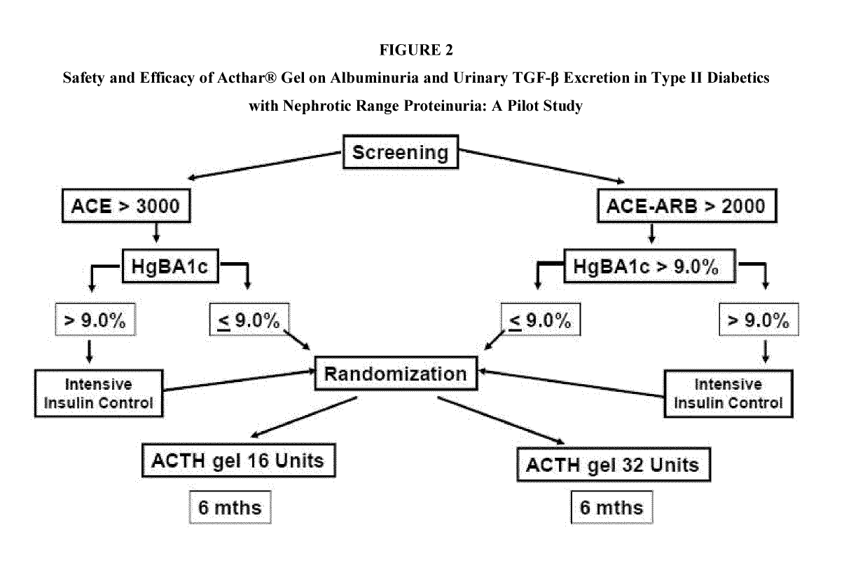

A pilot trial of sub-cutaneous Acthar® gel was performed in 15 patients with nephrotic range proteinuria secondary to over diabetic nephropathy. Of those 15 patients, 7 had formal renal biopsies confirming the presence of diabetic nodular sclerosis. FIG. 1 and FIG. 2 demonstrate an exemplary screening protocol and inclusion exclusion criteria for the study. Patients with type I or type II diabetic nephropathy and excreting greater than 3000 mg/24 hours on an ACE inhibitor or ARB were considered eligible for the study. Patients with an ACE/ARB or other protein lowering agent were required to have greater than 2000 mg of protein/24 hours. Patients with HgbAlc that were greater than 9.0% were ineligible for the study.

Patient Demographics:

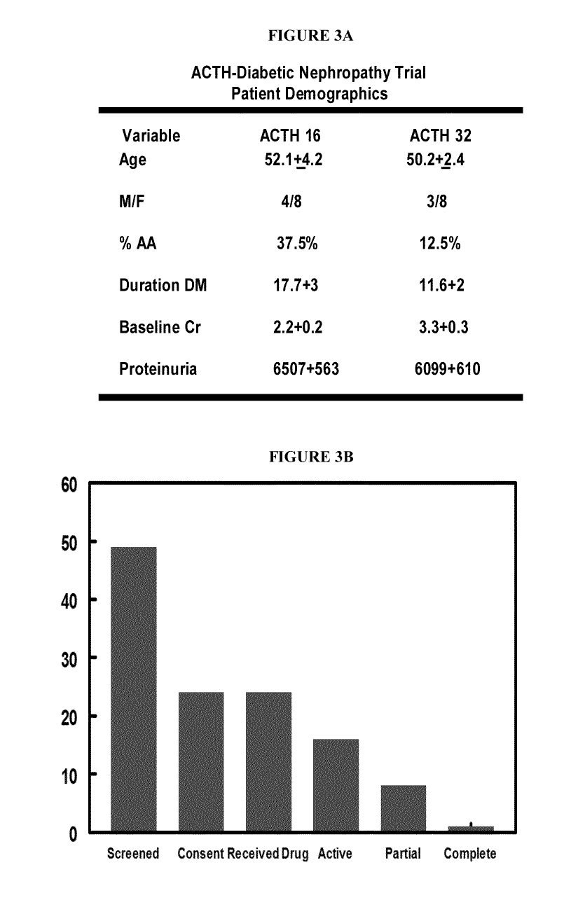

A total of 48 patients were screened for the trial with 25 giving informed consent and 23 electing not to participate. A total of 15 patients completed at least three months of therapy, with 14 completing the full six months of the protocol. As shown in FIG. 3A, the mean age of patients randomized to receive 16 or 32 units of SQ ACTH were comparable. The mean ages in the 16 and 32 unit groups were 52±4.2 and 50±2.4, respectively. The patients in both study groups exhibited nephrotic range proteinuria with the 16 and 32 unit groups having basal proteinuria of 6507±563 and 6099±610 mg/24 hours, respectively. FIG. 3B provides a breakdown of the number of patients throughout the screening process and trial.

ACTH Therapy: Response and Change in Urinary Proteinuria:

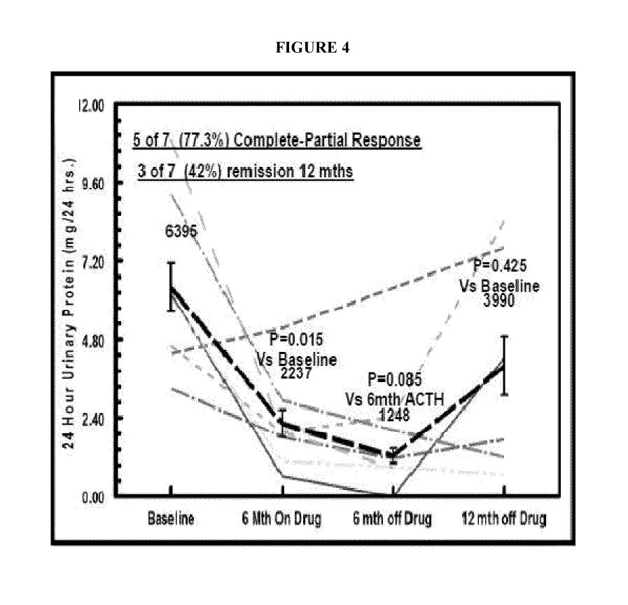

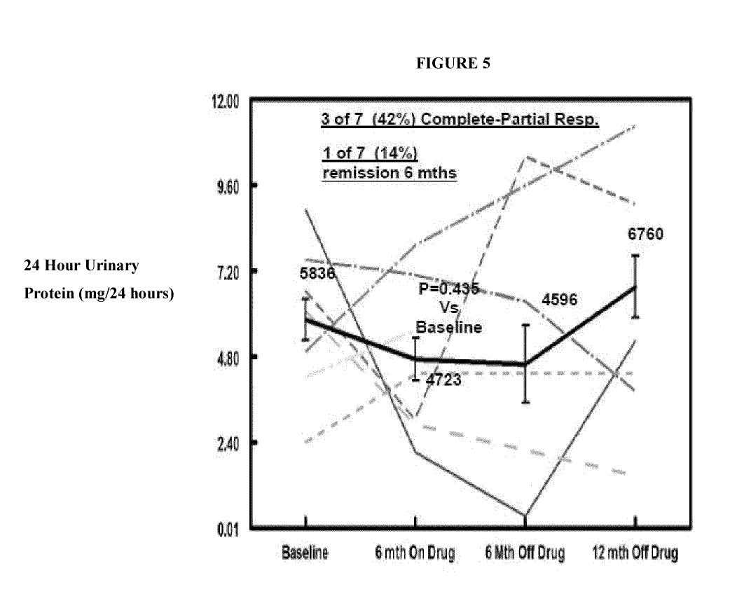

As shown in FIG. 4 and FIG. 5, the urinary protein levels were recorded at baseline and then after 6 months of treatment with 16 or 32 units of ACTH, respectively. More prolonged follow up data on urinary protein is recorded for 6 and 12 months off ACTH therapy.

As shown in FIG. 4, patients randomized to the 16 units dose clearly had a more pronounced effect with a higher proportion (77% vs. 42%) of patients achieving a complete or partial response. The mean proteinuria fell from a baseline level of 6395 to 2237 mg/24 hours after six months of ACTH treatment. Moreover, urinary protein among patients in the 16 unit cohort continued to fall during the 6 months after stopping ACTH injections (mean 1248 mg/24 hours). This data did not reach statistical significance.

As shown in FIG. 5, urinary protein was not altered by 32 units of ACTH. Moreover, urinary protein levels at six months failed to exhibit additional reduction as was noted with patients in the 16 unit cohort. The mechanism by which this lack of dose dependency was exhibited is unknown, but may involve threshold sensitivities to ACTH receptors.

Urinary VEGF Levels: Response to ACTH

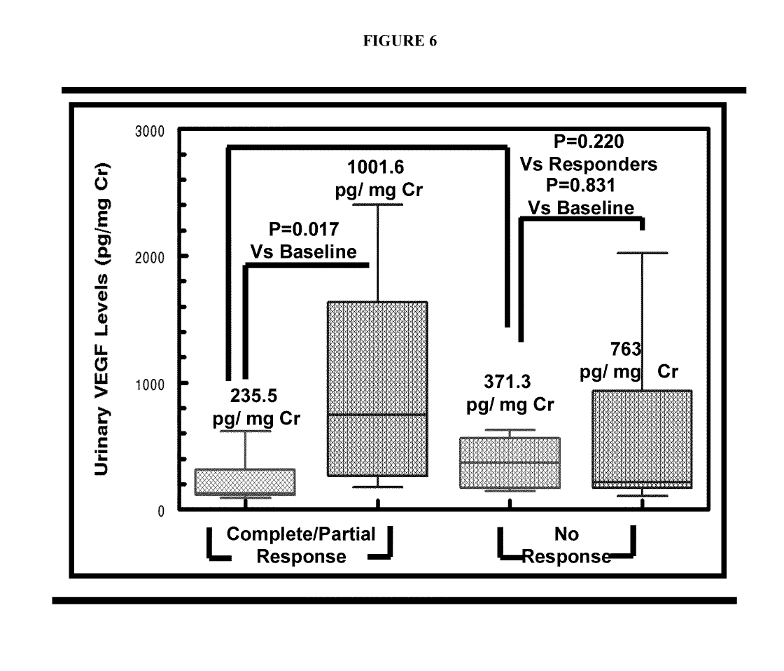

To determine whether the fall in urinary protein was related to a potential normalization of urinary VEGF, we measured pre-post urine levels of VEGF using an ELISA assay. As shown in FIG. 6, urinary VEGF among the responsive group rose by approximately 5-fold from a baseline value of 235.5 pg/mg Cr to a value of 1001.6 pg/mg Cr after 6 months of 16 units ACTH therapy.

The inventors do not currently have data on how long urinary VEGF levels will remain elevated following ACTH therapy. The observed rise in urinary protein within six months of stopping ACTH may correspond to a fall of urinary VEGF levels to pre-treatment levels. Thus, further doses of ACTH may be administered to a patient as needed to continue therapy.

Further, clinical monitoring of urinary VEGF levels may enable clinicians to determine both dose and duration of ACTH therapy in diabetics with nephrotic range proteinuria.

### Example 2

### Advanced Diabetic Nephropathy with Nephrotic Range Proteinuria: Long-Term Efficacy of Subcutaneous Adrenocorticotrophic Hormone (ACTH) Therapy on Proteinuria and Urinary Vascular Endothelial Growth Factor (VEGF) Levels

Activation of melanocortin receptor-1 (MClR) in podocytes and endothelium can lower proteinuria. The present example demonstrates that 6 months of ACTH gel reduces proteinuria in over 50% of patients with nephrotic diabetic nephropathy. The inventors investigated whether the reduction in proteinuria with ACTH gel involves alteration of VEGF expression.

Methods: A total of 14 patients with diabetic nephropathy and 3.0 gm proteinuria/24 hrs on ACE inhibitor alone or 2.0 gm/24 hrs on combination ACE/ARB were enrolled. All patients had eGFR≧20 mls/min and HgBAlc≦9%. Patients were randomized to ACTH gel (16 U or 32 U) subcutaneously daily for 6 months. Using a Luminex or ELISA assay, urinary VEGF and monocyte chemotactic protein-1 (MCP-1) were measured at baseline and after 6 months of ACTH gel. All urinary samples were normalized to Cr.

### Results:

| | ACTH gel 16 U | | |
|---|---|---|---|
| | | 6 months | 6 months |
| | Baseline | ACTH | post ACTH |
| Proteinuria | 63951 ± 735 | 2237 ± 399* | 1248 ± 235* |
| Urinary VEGF | 374 ± 107 | 1539 ± 403* | |
| Urinary MCP-1 | 569 ± 254 | 14011 ± 735 | |

*P < 0.05

ACTH gel (16 U) reduced proteinuria from 6395 to 2237 mg/24 hrs (P=0.015). After drug withdrawal (6 months), proteinuria further fell to 1248 mg/24 hrs (P=0.08). ACTH gel 16 U increased urinary VEGF (379 to 1539 pg/mg Cr) (P=0.04). Patients responding to ACTH gel therapy (>50% reduction in proteinuria) had lower baseline urinary VEGF than non-responders (388 vs. 689 pg/mg Cr)(P-0.022). Urinary MCP-1 tended to rise following treatment but did not reach statistical significance.

Conclusions: ACTH gel reduces urinary protein in diabetic nephropathy for up to 6 months after withdrawal of therapy. ACTH gel increased urinary VEGF 5-fold with a non-significant trend toward increased MCP-1. ACTH gel may represent a novel therapy for advanced diabetic nephropathy, and may act in part by restoring appropriate expression of VEGF.

### Example 3

### Pharmaceutical Compositions

### Example 2a

### Parenteral Composition

To prepare a parenteral pharmaceutical composition suitable for administration by intrathecal or intramuscular or intravenous or subcutaneous injection, 100 mg of a water-soluble salt of ACTH peptide or fragment, analog, complex or aggregate thereof, or any combination thereof, described herein, is dissolved in DMSO and then mixed with 10 mL of 0.9% sterile saline. A preservative and/or a stabilizer is optionally added to the mixture. The mixture is incorporated into a dosage unit form suitable for administration by injection.

### Example 2b

### Inhalation Composition

To prepare a pharmaceutical composition for inhalation delivery, 20 mg of ACTH peptide or fragment, analog, complex or aggregate thereof, or any combination thereof, is mixed with 50 mg of anhydrous citric acid and 100 mL of 0.9% sodium chloride solution. The mixture is incorporated into an inhalation delivery unit, such as a nebulizer, which is suitable for inhalation administration.

### Example 2c

### Rectal Gel Composition

To prepare a pharmaceutical composition for rectal delivery, 100 mg of ACTH peptide or fragment, analog, complex or aggregate thereof, or any combination thereof, is mixed with 2.5 g of methylcellulose (1500 mPa), 100 mg of methylparapen, 5 g of glycerin and 100 mL of purified water. The resulting gel mixture is then incorporated into rectal delivery units, such as syringes, which are suitable for rectal administration.

### Example 2d

### Topical Gel Composition

To prepare a pharmaceutical topical gel composition, 100 mg of ACTH peptide or fragment, analog, complex or aggregate thereof, or any combination thereof, is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 2e

### Oral Composition

To prepare a pharmaceutical composition for oral delivery, 100 mg of ACTH peptide or fragment, analog, complex or aggregate thereof, or any combination thereof, or a prodrug thereof, is mixed with 750 mg of starch. The mixture is incorporated into an oral dosage unit, such as a hard gelatin capsule, which is suitable for oral administration.

### Example 2f

### Nasal spray solution

To prepare a pharmaceutical nasal spray solution, 10 g of ACTH peptide or fragment, analog, complex or aggregate thereof, or any combination thereof, is mixed with 30 mL of a 0.05M phosphate buffer solution (pH 4.4). The solution is placed in a nasal administrator designed to deliver 100 μl of spray for each application.

### Example 4

### Assays

VEGF Assays

Methods by which human urinary VEGF may be identified and quantitated and are known in the art. Commercially available kits and antibodies may be used to detect the presence the protein, and further, quantitate the levels in a sample compared to a standard.

The Quantikine Human VEGF Immunoassay (R&D Systems, Inc.) is a solid phase ELISA designed to measure VEGF A and VEGF B.

It contains Sf 21-expressed recombinant human VEGF 165 and antibodies raised against the recombinant protein.

The first urine of the day (mid-stream) is collected aseptically, voided directly into a sterile container, and centrifuged to remove particulate matter. The samples are assayed immediately or aliquoted and stored at ˜−20° C. All urine samples are diluted at least a 2-fold dilution into Calibrator Diluent RD5L (1×).

Briefly, a monoclonal mouse anti-human antibody specific for VEGF A or VEGF B is pre-coated onto a microplate. Standards and samples are pipetted into the wells in duplicate or triplicate and any VEGF A or VEGF B present is bound by the immobilized antibody. After washing away any unbound substances, a biotinylated mouse anti-human antibody specific for VEGF A or VEGF B is added to the wells. Following a wash to remove any unbound antibody-enzyme reagent, a substrate solution is added to the wells and color develops in proportion to the amount of VEGF A or VEGF B bound in the initial step. The color development is stopped and the intensity of the color is measured.

The optical density of each well is determined within 30 minutes using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

The readings for each standard, control, and sample are averaged and the average zero standard optical density is subtracted.

A standard curve is created by reducing the data using computer software capable of generating a four parameter logistic (4-PL) curve-fit. As an alternative, a standard curve is constructed by plotting the mean absorbance for each standard on the y-axis against the concentration on the x-axis and drawing a best fit curve through the points on the graph. The data may be linearized by plotting the log of the VEGF A or VEGF B concentrations versus the log of the O.D. and the best fit line may be determined by regression analysis. Because samples have been diluted in the activation step prior to the assay, the measured concentrations are multiplied by the final dilution factor.

MCP-1 Assay

Methods by which human MCP-1 may be identified and quantitated and are known in the art. Commercially available kits and antibodies may be used to detect the presence the protein, and further, quantitate the levels in a sample compared to a standard.

The Quantikine Human MCP-1 Immunoassay (R&D Systems, Inc.) is a solid phase ELISA designed to measure MCP-1 in urine. It contains E. coli-expressed recombinant human MCP-1 and antibodies raised against the recombinant factor. The immunoassay has been shown to accurately quantitate recombinant human MCP-1.

The first urine of the day (mid-stream) is collected aseptically, voided directly into a sterile container, and centrifuged to remove particulate matter. The samples are assayed immediately or aliquoted and stored at ˜−20° C. All urine samples are diluted at least a 2-fold dilution into Calibrator Diluent RD5L (1×).

Briefly, a monoclonal antibody specific for MCP-1 is pre-coated onto a microplate. Standards and samples are pipetted into the wells in duplicate or triplicate and any MCP-1 present is bound by the immobilized antibody. After washing away any unbound substances, an enzyme-linked polyclonal antibody specific for MCP-1 is added to the wells. Following a wash to remove any unbound antibody-enzyme reagent, a substrate solution is added to the wells and color develops in proportion to the amount of MCP-1 bound in the initial step. The color development is stopped and the intensity of the color is measured.

The optical density of each well is determined within 30 minutes using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

The readings for each standard, control, and sample are averaged and the average zero standard optical density is subtracted.

A standard curve is created by reducing the data using computer software capable of generating a four parameter logistic (4-PL) curve-fit. As an alternative, a standard curve is constructed by plotting the mean absorbance for each standard on the y-axis against the concentration on the x-axis and drawing a best fit curve through the points on the graph. The data may be linearized by plotting the log of the MCP-1 concentrations versus the log of the O.D. and the best fit line may be determined by regression analysis. Because samples have been diluted in the activation step prior to the assay, the measured concentrations are multiplied by the final dilution factor.

TGF-β1 Assay

Methods by which human TGF-β1 may be identified and quantitated and are known in the art. Commercially available kits and antibodies may be used to detect the presence the protein, and further, quantitate the levels in a sample compared to a standard.

The Quantikine Human TGF-β1 Immunoassay (R&D Systems, Inc.) is a solid phase ELISA that can measure TGF-β1 in urine. Recombinant human TGF-β1 expressed by CHO cells is used as a standard for quantification. The assay is a quantitative sandwich enzyme immunoassay (ELISA) technique.

The first urine of the day (mid-stream) is collected aseptically, voided directly into a sterile container, and centrifuged to remove particulate matter. The samples are assayed immediately following activation or aliquoted and stored at ˜−20° C. To activate latent TGF-β1 to the immunoreactive form, solutions for acid activation (1N HCl) and neutralization (1.2 N NaOH/0.5 M HEPES) are prepared. Twenty (20) μL of 1 N HCl is added to 100 μL of the neutralized urine sample, mixed and incubated 10 minutes at room temperature. The acidified sample is neutralized by adding 20 μL of 1.2 N NaOH/0.5 M HEPES and mixing well.

Briefly, a monoclonal antibody specific for TGF-β1 is pre-coated onto a microplate. Standards, controls and samples are pipetted into the wells in duplicates or triplicates and any TGF-β1 present is bound by the immobilized antibody. After washing away any unbound substances, an enzyme-linked polyclonal antibody specific for TGF-β1 is added to the wells to sandwich the TGF-β1 immobilized during the first incubation. Following a wash to remove any unbound antibody-enzyme reagent, a substrate solution is added to the wells and color develops in proportion to the amount of TGF-β1 bound in the initial step. The color development is stopped and the intensity of the color is measured.

The optical density of each well is determined within 30 minutes using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

The readings for each standard, control, and sample are averaged and the average zero standard optical density is subtracted.

A standard curve is created by reducing the data using computer software capable of generating a four parameter logistic (4-PL) curve-fit. As an alternative, a standard curve is constructed by plotting the mean absorbance for each standard on the y-axis against the concentration on the x-axis and drawing a best fit curve through the points on the graph. The data may be linearized by plotting the log of the TGF-β1 concentrations versus the log of the O.D. and the best fit line may be determined by regression analysis. Because samples have been diluted in the activation step prior to the assay, the measured concentrations are multiplied by the final dilution factor.

While preferred embodiments have been shown and described herein, it will be obvious to those skilled in the art that such embodiments are provided by way of example only. Numerous variations, changes, and substitutions will now occur to those skilled in the art without departing from the disclosed embodiments. It should be understood that various alternatives to the embodiments described herein may be employed. It is intended that the following claims define the scope of the embodiments and that methods and structures within the scope of these claims and their equivalents be covered thereby.

### REFERENCES

1. USRDS: The United States Renal Data System. Am. J. Kidney Dis. 42 (Supplement-1), 1-230. 2003.
2. Anderson, G. N., Hagglundy, M., Nagaevay, 0., Fra\ddot{}ngsmyry, L., Petrovskaz, R., Mincheva-Nilssony, L., and Wikbergz, J. E. S. Quantitative Measurement of the Levels of Melanocortin Receptor Subtype 1, 2, 3 and 5 and Pro-Opio-Melanocortin Peptide Gene Expression in Subsets of Human Peripheral Blood Leucocytes. Scandinavian Journal of Immunology 61, 279-284. 2005.
3. Azar, S. T., Salti, I., and Zantout, M. S. Alterations in plasma transforming growth factor beta in normoalbuminuric type 1 and type 2 diabetic patients. J. Clin. Endocrinol. Metab. 85, 4680-4682. 2000.
4. Berg, A. L. and Arnadottir, M. ACTH-induced improvement in the nephrotic syndrome in patients with a variety of diagnoses. Nephrol Dial. Transplant 19, 1305-1307. 2004.
5. Berg, U. B., Torbjornsdotter, T. B., Jaremko, G., and Thalme, B. Kidney morphological changes in relation to long-term renal function and metabolic control in adolescents with IDDM. Diabetologia 41, 1047-1056. 1998.
6. Ellis, E. N., Steffes, M. W., Chavers, B., and Mauer, S. M. Observations of glomerular epithelial cell structure in patients with type I diabetes mellitus. Kidney Internal 32(786), 417. 1987.
7. Hohenstein, B, Daniel, C., Hausknecht, B., Boehmer, K., Riess, R., Amann, K. U., and Hugo, C. P. M. Correlation of enhanced thrombospondin-1 expression, TGFb-1 signalling and proteinuria in human type 2 diabetic nephropathy. Nephrol Dial. Transplant, 1-8. 2008.
8. Houlihan, C. A., Akdeniz, A., Tsalamandris, C., Cooper, M. E., Jerums, G., and Gilbert, R. E. Urinary transforming growth factor beta excretion in patients with hypertension, type II diabetes and elevated albumin excretion rate: Effect of angiotensin receptor blockade and sodium restriction. Diabetes Care 25(6), 1072-1077. 2002.
9. Johnson, E. W., Hughes, T. K., and Smith, E. M. ACTH receptor distribution and modulation among murine mononuclear leukocyte populations. J Biol. Regul. Homeost. Agents 15, 156-162. 2001.
10. Katavetin, P., Eiam-Ong, S., and Suwanwalaikorn, S. Pioglitazone reduces urinary protein and urinary transforming growth factor-beta excretion in patients with type 2 diabetes and overt nephropathy. J Med Assoc Thai. 89, 170-177. 2006.
11. Kuchenbauer, F., Hopfner, U., Stalla, J., Artz, E., Stalla, G. K., and Pa´ez-Pereda, M. Extracellular matrix components regulate ACTH production and proliferation in corticotroph tumor cells. Endocrinology 175(141), 148. 2001.
12. Matos, J. P., de Lourdes Rodrigues, M., and Ismerim, V. L. Effects of dual blockade of the rennin angiotensin system in hypertensive type 2 diabetic patients with nephropathy. Clin Nephrol. 64, 180-189. 2005.
13. Meyer, T. W., Bennett, P. H., and Nelson, R. G. Podocyte number predicts long-term urinary albumin excretion in Pima Indians with type II diabetes and microalbuminuria. Diabetologia 42(1341), 1344. 1999.
14. Steffes M W, Basgen, J. M., Schmidt, D., and McCrery, R. Glomerular cell number in normal subjects and in type 1 diabetic patients. Kidney Int 59, 2104-2113. 2001.
15. Steffes M W, Leffert, J. D., Basgen, J. M., Brown, D. M., and Mauer, S. M. Epithelia cell foot process width in intact and uninephrectomized diabetic and nondiabetic rats. Lab Invest 43, 225-230. 1998.
16. Steffes, M. W., Schmidt, D., McCrery, R., and Basgen, J. M. International Diabetic Nephropathy Study Group. Glomerular cell number in normal subjects and in Type 1 diabetic patients. Kidney Internal 59(2104), 2113. 2008.
17. Wu, D. T., Bitzer, M., Wenjun, J., Mundel, P., and Bottinger, E. P. TGF-Concentration Specifies Differential Signaling Profiles of Growth Arrest/Differentiation and Apoptosis in Podocytes. J. Am. Soc. Nephrol. 16, 3211-3221. 2005.
18. Mallet, C., Feraud, 0., Ouengue-Mbele, G., Gaillard, I., Sappay, N., Vittet, D., Vilgrain, I. Differential expression of VEGF receptors in adrenal atrophy induced by dexamethasone: a protective role of ACTH. Am. J. Physiol. Endocrinol. Metab. 284, E156-E167. 2003.
19. Qiu, Y., Ferguson, J., Sebastian Oltean, S., Neal, C. R., Kaura, A., Bevan, H., Emma Wood, Sage, L. M. Lanati, S., Nowak, D. G, Salmon, A. H. J., David Bates, D., and Harper, S. J. Overexpression of VEGF165b in Podocytes Reduces Glomerular Permeability. J Am Soc Nephrol 21: 1498-1509, 2010.
20. Cui, T. G., Foster, R. R., Saleem, M., Mathieson, P. W., Gillatt, D. A., Bates, D. O., Harper, S. J. Differentiated human podocytes endogenously express an inhibitory isoform of vascular endothelial growth factor (VEGF165b) mRNA and protein. Am J Physiol Renal Physiol 286:F767-F773, 2004.
21. Mandriota, S. J. Menoud, P. A., Michael S. Pepper, M. S. Transforming Growth Factor b1 Down-regulates Vascular Endothelial Growth Factor Receptor 2/flk-1 Expression in Vascular Endothelial Cells J. Biol. Chem. 271 (1), 11500-11505, 1996.
22. Amann B, Tinzmann R, Angelkort B. ACE inhibitors improve diabetic nephropathy through suppression of renal MCP-1. Diabetes Care 26: 2421-2425, 2003.
23. Wada T, Furuichi K, Sakai N, Iwata Y, Yoshimoto K, Shimizu M, Takeda S I, Takasawa K, Yoshimura M, Kida H, Kobayashi K I, Mukaida N, Naito T, Matsushima K, Yokoyama H. Up-regulation of monocyte chemoattractant protein-1 in tubulointerstitial lesions of human diabetic nephropathy. Kidney Int 58: 1492-1499, 2000.
24. Wikberg J E S, Muceniece R, Mandrika I, et al. New aspects on the melanocortins and their receptors. Pharmacol Res 2000; 42: 393-420.
25. Sugimoto, H., Hamano, Y., Charytan, D, Cosgrove, Kieran, M. D., Sudhakar, A. Kalluri, R. Neutralization of Circulating Vascular Endothelial Growth Factor (VEGF) by Anti-VEGF Antibodies and Soluble VEGF Receptor 1 (sFlt-1) Induces Proteinuria. J. Biol. Chem. 278(15), 12605-12608, 2003
26. Nakagawa, T. Uncoupling of the VEGF-endothelial nitric oxide axis in diabetic nephropathy: an explanation for the paradoxical effects of VEGF in renal disease. Am J Physiol Renal Physiol 292:F1665-F1672, 2007.

What is claimed is:

1. A method of identifying a patient for treatment with ACTH(1-39), a fragment thereof, or an analog thereof, and/or managing the dose, interval and/or duration of ACTH treatment in the long-term management of diabetic nephropathy or nephrotic range proteinuria in a patient comprising:
measuring levels of urinary VEGF 121, VEGF 165 and MCP-1 in one or more urine samples obtained from said patient; and
adjusting the dose, interval and/or duration of ACTH(1-39), a fragment thereof, or an analog thereof administered to said patient based on the levels of said VEGF 121, VEGF 165 and MCP-1.

2. The method of claim 1, wherein if
the concentration of urinary VEGF 121 is greater than about 200 pg/mg Cr;
the concentration of urinary VEGF 165 is greater than about 160 pg/mg Cr;
the concentration of urinary MCP-1 is less than about 1000 mg/24 hours or greater than about 0.25 ng/mg; and
the ratio of VEGF 121 to VEGF 165 is below about 0.80, a patient begins treatment, the dose is increased or the duration of treatment is increased.

3. The method of claim 1, wherein if
the concentration of urinary VEGF 121 is less than about 200 pg/mg Cr;
the concentration of urinary VEGF 165 is less than about 160 pg/mg Cr;
the concentration of urinary MCP-1 is greater than about 1000 mg/24 hours or less than about 0.25 ng/mg; and
the ratio of VEGF 121 to VEGF 165 is above about 0.80, a patient ceases treatment, the dose is decreased or the duration of treatment is decreased.

4. The method of claim 1, wherein the dose of ACTH(1-39), a fragment thereof, or an analog thereof, is decreased if a patient is identified as successfully responding to treatment.

5. The method of claim 1, wherein ACTH(1-39), a fragment thereof, or an analog thereof, is administered as Acthar® gel.

6. The method of claim 1, wherein said urine samples are obtained pre-treatment and post-treatment.

7. The method of claim 1, further comprising measuring the level urinary TGF-β, urinary creatine, proteinuria or a combination thereof.

8. A method of decreasing glomerular permeability in a patient, comprising:
measuring levels of urinary VEGF 121, VEGF 165 and MCP-1 in one or more urine samples obtained from said patient;
wherein if the concentration of urinary VEGF 121 is greater than about 200 pg/mg Cr, the concentration of urinary VEGF 165 is greater than about 160 pg/mg Cr; and the concentration of urinary MCP-1 is greater than about 0.25 ng/mg or less than about 1000 mg/24 hours;
the dose and/or duration of ACTH(1-39), a fragment thereof, or an analog thereof, administered to said patient is increased.

9. The method of claim 8, wherein ACTH(1-39), a fragment thereof, or an analog thereof, is administered as Acthar® gel.

10. The method of claim 8, wherein said urine samples are obtained pre-treatment and post-treatment.

11. The method of claim 8, further comprising measuring the level urinary TGF-β, urinary creatine, proteinuria or a combination thereof.

12. The method of claim 8, wherein rising levels of VEGF 121 and MCP-1 and decreasing levels of VEGF 165 as biomarkers indicate increasing glomerular permeability, and the dose and/or duration of ACTH(1-39), a fragment thereof, or an analog thereof, treatment is increased.

13. The method of claim 8, wherein decreasing levels of VEGF 121 and MCP-1 and increasing levels of VEGF 165 as biomarkers indicate decreasing glomerular permeability, and the dose and/or duration of ACTH(1-39), a fragment thereof, or an analog thereof, treatment is decreased.

14. A method of decreasing proteinuria in a patient, comprising
measuring levels of urinary VEGF 121, VEGF 165 and MCP-1 in one or more urine samples obtained from said patient;
wherein if the ratio of VEGF 121 to VEGF 165 falls below 0.75, proteinuria is diagnosed as increasing; and the dose and/or duration of ACTH(1-39), a fragment thereof, or an analog thereof, administered to said patient is increased.

15. The method of claim 14, wherein ACTH(1-39), a fragment thereof, or an analog thereof, is administered as Acthar® gel.

16. The method of claim 14, wherein said urine samples are obtained pre-treatment and post-treatment.

17. The method of claim 14, further comprising measuring the level urinary TGF-β, urinary creatine, proteinuria or a combination thereof.

18. A method of identifying a patient for treatment with ACTH(1-39), a fragment thereof, or an analog thereof, and/or increasing the dose and/or duration of ACTH(1-39), a fragment thereof, or an analog thereof treatment in the long-term management of diabetic nephropathy or nephrotic range proteinuria in a patient comprising:
measuring levels of urinary VEGF 121 of between about 200 pg/ml and 800 pg/ml, urinary levels of VEGF 165 of between about 180 pg/ml and 2000 pg/ml, and urinary levels of MCP-1 of between about 0.25 ng/mg or about 400 mg/24 hours to about 0.4 ng/mg or 1000 mg/24 hours in one or more samples obtained from said patient;
identifying a ratio of VEGF 121 to VEGF 165 of below about 0.80; and
beginning treatment of said patient or increasing the dose and/or duration of ACTH(1-39), a fragment thereof, or an analog thereof administered to said patient based on the levels of said VEGF 121, VEGF 165 and MCP-1.

19. The method of claim 18, wherein ACTH(1-39), a fragment thereof, or an analog thereof, is administered as Acthar® gel.

20. A method of identifying a patient for treatment with ACTH(1-39), a fragment thereof, or an analog thereof, and/or increasing the dose and/or duration of ACTH(1-39), a fragment thereof, or an analog thereof, treatment in the long-term management of diabetic nephropathy or nephrotic range proteinuria in a patient comprising:
identifying levels of urinary VEGF 121 of least 200 pg/ml, urinary levels of VEGF 165 of at least 180 pg/ml and urinary levels of MCP-1 of less than about 1000 mg/24 hours or about 0.25 ng/mg in one or more samples obtained from said patient and
beginning treatment of said patient or increasing the dose and/or duration of ACTH(1-39), a fragment thereof, or an analog thereof, administered to said patient based on the levels of said VEGF 121, VEGF 165 and MCP-1.

21. The method of claim 20, wherein ACTH(1-39), a fragment thereof, or an analog thereof, is administered as Acthar® gel.

22. A method of identifying a patient for cessation of treatment with ACTH(1-39), a fragment thereof, or an analog thereof, and/or decreasing the dose and/or duration of ACTH(1-39), a fragment thereof, or an analog thereof, treatment in the long-term management of diabetic nephropathy or nephrotic range proteinuria in a patient comprising:
measuring levels of urinary VEGF 121 of below about 200 pg/ml, urinary levels of VEGF 165 of below about 180 pg/ml, and urinary levels of MCP-1 of greater than about 1000 mg/24 hours or below about 0.25 ng/mg in one or more samples obtained from said patient;
measuring a ratio of VEGF 121 to VEGF 165 of above about 0.80; and
ceasing treatment of said patient or decreasing the dose and/or duration of ACTH(1-39), a fragment thereof, or an analog thereof, administered to said patient based on the levels of said VEGF 121, VEGF 165 and MCP-1.

23. The method of claim 22, wherein ACTH(1-39), a fragment thereof, or an analog thereof, is administered as Acthar® gel.

24. A method of identifying a patient for treatment with ACTH(1-39), a fragment thereof, or an analog thereof, and/or increasing the dose and/or duration of ACTH(1-39), a fragment thereof, or an analog thereof treatment in the long-term management of diabetic nephropathy or nephrotic range proteinuria in a subject comprising:
measuring levels of urinary VEGF 121, VEGF 165 and MCP-1 in urine samples obtained from said patient, wherein if the ratio of VEGF 121 to VEGF 165 is below about 0.80 and if the level of urinary MCP-1 is less than about 1000 mg/24 hours or above about 0.25 ng/mg, the patient is identified for further treatment; and
beginning treatment of said patient or increasing the dose and/or duration of ACTH(1-39), a fragment thereof, or an analog thereof, treatment.

25. The method of claim 24, wherein ACTH(1-39), a fragment thereof, or an analog thereof, is administered as Acthar® gel.

26. A method of identifying a patient for cessation of treatment with ACTH(1-39), a fragment thereof, or an analog thereof, and/or decreasing the dose and/or duration of ACTH(1-39), a fragment thereof, or an analog thereof, treatment in the long-term management of diabetic nephropathy or nephrotic range proteinuria in a subject comprising:
measuring levels of urinary VEGF 121, VEGF 165 and MCP-1 in urine samples obtained from said patient, wherein if the ratio of VEGF 121 to VEGF 165 is above about 0.80 and if the level of urinary MCP-1 is greater than about 1000 mg/24 hours or below about 0.25 ng/mg, the patient is identified as needing reduced treatment; and
ceasing treatment of said patient or decreasing the dose and/or duration of ACTH(1-39), a fragment thereof, or an analog thereof treatment.

27. The method of claim 26, wherein the patient is identified as being in remission.

28. The method of claim 26, wherein ACTH(1-39), a fragment thereof, or an analog thereof, is administered as Acthar® gel.

29. A system for identifying a patient for treatment with ACTH(1-39), a fragment thereof, or an analog thereof, and/or managing the dose, interval and/or duration of ACTH treatment in the long-term management of diabetic nephropathy or nephrotic range proteinuria in a patient comprising:
Antibodies that specifically bind to urinary VEGF 121, VEGF 165 and MCP-1;
An optical density microplate reader; and
a composition comprising ACTH(1-39), a fragment thereof, or an analog thereof.

30. The system of claim 29, further comprising: an optionally networked computer processing device configured to perform executable instructions; and a computer program, the computer program comprising a software module executed by the computer processing device to apply a model or algorithm for analyzing the urinary levels of VEGF 121, VEGF 165 and MCP-1 in the sample.

31. A computer-implemented system comprising:
a. a computer comprising: a processor, an operating system configured to perform executable instructions, and a memory device;
b. a computer program including instructions executable by the computer, the program comprising:
i. a software module configured to receive data indicating levels of urinary VEGF 121, VEGF 165 and MCP-1 in one or more urine samples obtained from a patient, the patient in need of long-term management of diabetic nephropathy or nephrotic range proteinuria;
ii. a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and
iii. a software module configured to generate a report comprising a recommendation for an adjustment to the dose, interval and/or duration of ACTH(1-39), a fragment thereof, or an analog thereof administered to the patient.

32. The system of claim 31, wherein the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

33. The system of claim 31, wherein the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.

34. A non-transitory computer-readable storage media encoded with a computer program including instructions executable by a processor, the program comprising:
a. a software module configured to receive data indicating levels of urinary VEGF 121, VEGF 165 and MCP-1 in one or more urine samples obtained from a patient, the patient in need of long-term management of diabetic nephropathy or nephrotic range proteinuria;
b. a software module configured to apply a model or algorithm for recommending adjustment to the dose, interval and/or duration of ACTH(1-39), a fragment thereof, or an analog thereof administered to the patient based on the levels of said VEGF 121, VEGF 165 and MCP-1; and
c. a software module configured to generate a report comprising a recommendation for an adjustment to the dose, interval and/or duration of ACTH(1-39), a fragment thereof, or an analog thereof administered to the patient.

35. The media of claim 34, wherein the model or algorithm recommends the patient begin treatment, the dose is increased, or the duration of treatment is increased if: the concentration of urinary VEGF 121 is greater than about 200 pg/mg Cr; the concentration of urinary VEGF 165 is greater than about 160 pg/mg Cr; the concentration of urinary MCP-1 is less than about 1000 mg/24 hours or greater than about 0.25 ng/mg; and the ratio of VEGF 121 to VEGF 165 is below about 0.80.

36. The media of claim 34, wherein the model or algorithm recommends the patient cease treatment, the dose is decreased, or the duration of treatment is decreased if: the concentration of urinary VEGF 121 is less than about 200 pg/mg Cr; the concentration of urinary VEGF 165 is less than about 160 pg/mg Cr; the concentration of urinary MCP-1 is greater than about 1000 mg/24 hours or less than about 0.25 ng/mg; and the ratio of VEGF 121 to VEGF 165 is above about 0.80.
